(19) United States
(12) Patent Application Publication
(10) Pub. No.: US 2008/0063704 A1
(43) Pub. Date: Mar. 13, 2008

(54) Stable gabapentin compositions

(75) Inventor: Austen John Woolfe, Essex (GB)

Correspondence Address: LERNER, DAVID, LITTENBERG,;KRUMHOLZ & MENTLIK, 600 SOUTH AVENUE WEST, WESTFIELD, NJ, 07090, US

(73) Assignee: Norton Healthcare Ltd., London (GB)

(21) Appl. No.: 10/585,912

(22) PCT Filed: Jan. 15, 2005
(86) PCT No.: PCT/US05/01423

§ 371 Date: Sep. 11, 2007

(30) Foreign Application Priority Data
Jan. 16, 2004 (GB) 0400993.2

(51) Int. Cl. A61K 31/192; A61K 9/20

(52) U.S. Cl. 424/464; 514/561

(57) ABSTRACT

The present invention provides a pharmaceutical composition of gabapentin wherein lactam level remains below 0.5% even after more than two years of storage at 25 to 30° C. and 60% relative humidity.

### FIELD OF INVENTION

This invention is concerned with stable gabapentin compositions. More particularly, this invention is concerned with pharmaceutical compositions comprising a therapeutically effective amount of gabapentin and an excipient which is not detrimental to the long-term stability of gabapentin.

### BACKGROUND OF THE INVENTION

Gabapentin (I) and its pharmaceutically acceptable salts have been used for a number of years for the treatment of cerebral disorders such as epilepsy, fainting attacks, hypokinesis and cranial traumas, has been known for many years, for example as disclosed in U.S. Pat. No. 4,024,175, U.S. Pat. No. 4,087,544 and U.S. Pat. No. 4894476.

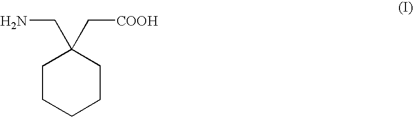

U.S. Pat. No. 6,054,482 discloses that the preparation and long-term storage of gabapentin and its pharmaceutically acceptable salts present problems since (i) during the preparation the compounds show considerable variations without apparent reason; (ii) very pure gabapentin, when stored long term, shows differing stabilities; and (iii) a toxic lactam (II) is formed when the gabapentin degrades. Pharmaceutically acceptable gabapentin compositions must comprise no more than 0.5% by weight of this toxic lactam compound.

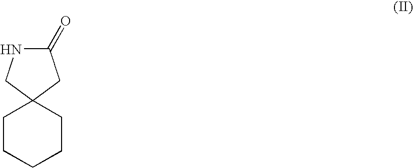

To combat lactam formation and provide long-term stability in pharmaceutical compositions, US-A-6054482 teaches that the following procedures must be maintained:

1. The active gabapentin materials must be prepared as highly purified, non-derivatized free amino acids, for example from the corresponding hydrochloride by ion exchange. The proportion of remaining hydrochloride admixtures, or anions of other mineral acids, should thereby not exceed 20 ppm;

2. To suppress the formation of toxic lactam, a particular excipient must be used.

Under the above storage conditions generally applicable for medicaments, toxic lactam formation does not increase within a period of time of 1 year after production of the pharmaceutical composition or of the active material by more than 0.2% by wt and preferably 0.1% by wt, based on the weight of the pure active material.

In addition, U.S. Pat. No. 6,054,482 provides a specific list of excipient materials which do not influence the stability of the active gabapentin compound when the proportion of mineral acid does not exceed 20 ppm. These are: hydroxypropylmethyl cellulose, polyvinyl pyrrolidone, crospovidone, poloxamer 407, poloxamer 188, sodium starch glycolate, copolyvidone, maize starch, cyclodextrin, lactose, talc as well as co-polymers of dimethylamino-methacrylic acid and neutral methacrylic acid ester. It also provides a specific list of excipient materials which reduce the stability of the active gabapentin compounds: these are modified maize starch, sodium croscarmellose, glycerol behenic acid ester, methacrylic acid co-polymers (types A and C), anion exchangers, titanium dioxide, and silica gels such as Aerosil 200.

U.S. Pat. No. 6,531,509 discloses that the long-term stability of pharmaceutical compositions based on active gabapentin compounds is not affected by the nature of the excipient materials disclosed in U.S. Pat. No. 6,054,482 provided that the amount of mineral acid anion in the composition is in excess of 20 ppm.

It is desired that pharmaceutical compositions are very stable, so that they can be offered for sale with very long shelf lives. It is also preferred that stable, pharmaceutically active compositions are not limited to pre-determined amounts of mineral acid anion.

Neither U.S. Pat. No. 6,054,482 nor U.S. Pat. No. 6,531,509 suggest that the compositions disclosed therein may be stable for very long periods e.g. for at least two years, irrespective of the amount of mineral acid anion or excipient.

We now report pharmaceutically effective gabapentin compositions which are stable for very long periods of time i.e. compositions to contain less than 0.5% lactam after at least two years of storage at 25° C. and 60% atmospheric humidity. Stability results for different durations and temperatures are included hereinafter.

### SUMMARY OF THE INVENTION

Accordingly, the present invention provides a stable pharmaceutical composition of gabapentin, stable in storage for extended periods, under conditions selected from the ranges consisting of: storage for at least 3 years at 25° C. and 60% relative humidity, storage for at least 2 years at 30° C. and 60% relative humidity, and storage for at least 6 months at 40° C. and 75% relative humidity.

According to another aspect of the present invention there is provided a pharmaceutical composition comprising gabapentin and microcrystalline cellulose as the sole excipient. Alternatively, the composition also comprises a lubricant, such as magnesium stearate.

According to another aspect of the present invention there is provided a pharmaceutical composition comprising gabapentin and microcrystalline cellulose as the sole diluent.

Preferably the composition is in the form of a capsule.

Preferably the capsule comprises hard gelatin.

Preferably the composition of the hard gelatin capsule further comprises one or more of: methyl hydroxy benzoate; propyl hydroxyl benzoate, titanium oxide, yellow iron oxide, red iron oxide, in any suitable combinations. The composition of the gelatin capsule may also contain purified water.

According to another aspect of the present invention there is provided a pharmaceutical composition comprising gabapentin and excipients including microcrystalline cellulose and magnesium stearate. Preferably, these are the only excipients, in which case microcrystalline cellulose is regarded as a diluent and magnesium stearate as a lubricant.

According to another aspect of the present invention there is provided a pharmaceutical composition comprising gabapentin and one or more of the following as a diluent: dibasic calcium phosphate; tribasic calcium phosphate; calcium sulphate; mannitol; microcrystalline cellulose; starch; and lactose.

According to another aspect of the present invention there is provided a pharmaceutical composition comprising gabapentin and one or more of the following as a lubricant: magnesium stearate; stearic acid; and colloidal silicon dioxide.

According to another aspect of the present invention there is provided a pharmaceutical composition comprising gabapentin and sodium lauryl sulphate.

According to another aspect of the present invention there is provided a pharmaceutical composition comprising gabapentin, the composition further comprising:

(i) microcrystalline cellulose;

(ii) magnesium stearate; and

(iii) sodium lauryl sulphate.

In certain embodiments, this composition also contains colloidal silicon dioxide.

The content of mineral acid anions may be less than 70 ppm, and more preferably less than 50 ppm or 30 ppm. These ranges are not intended to be limiting and contents outside these ranges can be used.

### DETAILED DESCRIPTION OF THE PREFERRED EMBODIMENTS

### Table Titles

Table 1: Composition of the proprietary medical product

Table 2: Stability Specification and routine tests for Gabapentin Capsules

Table 3: Details of batches put on stability

Table 4: Stability of Gabapentin Capsules 100 mg stored in PVC/PVdC/Aluminium packs (Batch Number GBC-I/100) stored at 25° C.±2° C./60%±5% RH

Table 5: Stability of Gabapentin Capsules 100 mg stored in PVC/PVdC/Aluminium packs (Batch Number GBC-I/100) stored at 30° C±2° C./60%±5% RH

Table 6: Stability of Gabapentin Capsules 100 mg stored in PVC/PVdC/Aluminium packs (Batch Number GBC-I/100) stored at 40° C.±2° C./ 75%±5% RH

Table 7: Stability of Gabapentin Capsules 100 mg stored in PVC/PVdC/Aluminium packs (Batch Number GBC-II/100) stored at 25° C.±2° C./60%±5% RH

Table 8: Stability of Gabapentin Capsules 100 mg stored in PVC/PVdC/Aluminium packs (Batch Number GBC-II/100) stored at 30° C.±2° C./60%±5% RH

Table 9: Stability of Gabapentin Capsules 100 mg stored in PVC/PVdC/Aluminium packs (Batch Number GBC-II/100) stored at 40° C.±2° C./75%±5% RH

Table 10: Stability of Gabapentin Capsules 300 mg stored in PVC/PVdC/Aluminium packs (Batch Number GBC-I/300) stored at 25° C.±2° C./60%±5% RH

Table 11: Stability of Gabapentin Capsules 300 mg stored in PVC/PVdC/Aluminium packs (Batch Number GBC-I/300) stored at 30° C.±2° C./60%±5% RH

Table 12: Stability of Gabapentin Capsules 300 mg stored in PVC/PVdC/Aluminium packs (Batch Number GBC-II/300) stored at 30° C.±2° C./60%±5% RH

Table 13: Stability of Gabapentin Capsules 300 mg stored in PVC/PVdC/Aluminium packs (Batch Number GBC-II/300) stored at 25° C.±2° C./ 60%±5% RH

Table 14: Stability of Gabapentin Capsules 400 mg stored in PVC/PVdC/Aluminium packs (Batch Number GBC-I/400) stored at 25° C.±2° C./60%±5% RH

Table 15: Stability of Gabapentin Capsules 400 mg stored in PVC/PVdC/Aluminium packs (Batch Number GBC-I/400) stored at 30° C.±2° C./60%±5% RH

Table 16: Stability of Gabapentin Capsules 400 mg stored in PVC/PVdC/Aluminium packs (Batch Number GBC-I1/400) stored at 40° C.±2° C./75%±5% RH

Table 17: Stability of Gabapentin Capsules 400 mg stored in PVC/PVdC/Aluminium packs (Batch Number GBC-II/400) stored at 25° C.±2° C./60%±5% RH

Table 18: Stability of Gabapentin Capsules 400 mg stored in PVC/PVdC/Aluminium packs (Batch Number GBC-II1/400) stored at 30° C.±2 ° C./60%±5% RH

Table 19: Stability of Gabapentin Capsules 400 mg stored in PVC/PVdC/Aluminium packs (Batch Number GBC-II/400) stored at 40° C.±2° C./75%±5% RH

Table 20: Stability of Neurontin® Capsules 400 mg stored in PVC/PVdC/Aluminium packs stored at 25° C.±2° C. /60%±5% RH

Table 21: Stability of Neurontin® Capsules 400 mg stored in PVC/PVdC/Aluminium packs stored at 30° C.±2° C./60%±5% RH

Table 22: Stability of Neurontin® Capsules 400 mg stored in PVC/PVdC/Aluminium packs stored at 40° C.±2° C./75%±5% RH

Table 23: Excipients used in the pre-formulation studies

Table 24: Trial blends for pre-formulation studies.

Table 25: Pre-formulation studies, Batch Number: GD

Table 26: Pre-formulation studies, Batch Number: GD2

Table 27: Pre-formulation studies, Batch Number: GD3

Table 28: Pre-formulation studies, Batch Number: GD4

Table 29: Pre-formulation studies, Batch Number: GD5

Table 30: Pre-formulation studies, Batch Number: GD6

Table 31: Pre-formulation studies, Batch Number: GD7

Table 32: Pre-formulation studies, Batch Number: GL1

Table 33: Pre-formulation studies, Batch Number: GL2

Table 34: Pre-formulation studies, Batch Number: GL3

Table 35: Pre-formulation studies, Batch Number: GS1

Table 36: Pre-formulation studies, Blend I

Table 37: Pre-formulation studies, Blend II

Table 38: Pre-formulation studies, Neurontin® Capsules 400 mg, Batch Number: 015077

Table 39: Pre-formulation studies, Drug Substance, Gabapentin Lot number: R 90562

The invention shall now be described further by way of exemplification.

### Experimental Protocols

HPLC Assay for Related Substances

Chromatographic Conditions

### Column

YMC—ODS—AQ, 5 μm, 250 mm×4.6 or equivalent

### Column Temperature

Ambient

### Mobile Phase

0.025 M Potassium Phosphate Monobasic (pH 6.0): Methanol (70:30)

### Detector

UV at 210 nm

### Flow Rate

10 ml/minute

### Injection Volume

50 μl

### Run Time

10 minutes or as appropriate for Standard Solution

15 minutes or as appropriate for Resolution Solution

70 minutes or as appropriate for Lactam Marker, Test Solution and Diluent Solutions.

### Needle Wash Solution

Water:methanol (70:30)

### Mobile Phase Preparation

0.025M potassium phosphate, monobasic (KH₂PO₄) buffer solution pH 6.0: Weight about 6.8 g of potassium phosphate, monobasic (KH₂PO₄) and dissolve in about 1800 ml of water. Adjust pH of the solution to 6.0 (±0.05) using 1 N Sodium Hydroxide solution. Add sufficient water to make 2000 ml and mix well.

Mobile Phase: Mix 1400 ml of 0.025 M potassium phosphate, monobasic (KH₂PO₄) buffer solution pH 6.0 with 600 ml methanol, filter and degas.

### Sample Solution Preparation

Weigh 20 intact capsules. Empty the capsules as completely as possible into a suitable container. Clean and weigh the empty capsule shells and determine the average capsule filled weight. Mix thoroughly the combined contents of the capsules.

Weigh accurately amount of powder equivalent to 600 mg Gabapentin into a 50 ml volumetric flask. Add about 30 ml of mobile phase and sonicate for 10 minutes with intermittent shaking to disperse the powder. Shake for 30 minutes. Dilute to volume with mobile phase and mix well. Filter the solution.

### Standard Solution Preparation

Stock Standard Solution

Weigh accurately about 25 mg Gabapentin R.S. and transfer to 50 ml volumetric flask. Add to it about 25 ml mobile phase. Sonicate to dissolve, make up the volume with mobile phase.

Working Standard Solution

Pipette out 6 ml of the solution from Standard Stock Solution into 50 ml volumetric Flask. Dilute to volume with mobile phase and mix well.

### Resolution Solution Preparation

Resolution stock solution (C.A.M)

Weigh accurately about 12.5 mg of C.A.M., dissolved and dilute to 25 ml with methanol

Note: Store under refrigeration for future use. The solution may be used as long as a peak due to C.A.M. is clearly visible in the chromatogram.

### Resolution Working Solution

Pipette out 6 ml of Standard Stock Solution and 6 ml of C.A.M. Stock Solution and dilute to 50 ml with mobile phase.

### Lactam Marker Solution Preparation

Lactam stock solution

Weigh accurately about 12.5 mg lactam, dissolve and dilute to 25 ml with methanol.

Note: Store under refrigeration for future use. The solution nay be used as long as the lactam peak is clearly seen.

### Lactam Working Solution

Pipette 6 ml of Lactam Stock solution and dilute to 50 ml with mobile phase.

### Preparation of Methyl Parabens Marker Solution

Weigh accurately about 25 mg of methyl parabens and dissolve and dilute to 50 ml with mobile phase. Pipette 5 ml of solution into a 50 ml volumetric flask and make up to volume with mobile phase. Further pipette 5 ml and dilute to 50 ml with mobile phase.

### System Suitability

System suitability test solution: Inject 50 μl of Resolution Solution into the equilibrated chromatograph. Calculate the system suitability requirements. Gabapentin peak has retention time of about 6 minutes. C.A.M. has retention time of about 10 minutes

The resolution between Gabapentin and C. A. M. peaks is NLT 4.5

The tailing factor (T), determined from the Gabapentin peak is NMT 2.0%. Perform 6 replicate injections of 50 μl of Working Standard Solution. The System precision is acceptable if the RSD of 6 replicate standard injections is NMT 5.0%

### Procedure

Separately inject 50 μl of the mobile phase, Standard Solution, lactam marker solution, Methyl Parabens marker solution and Test Solutions into the Chromatograph. Measure the responses of the major peaks.

Calculate the content of impurity lactam: single largest individual/unidentified impurity/degradant and total impurities/degradant.

Note: Identify the peak due to methyl parabens based upon the retention time in the chromatogram of the Methtyl Parabens marker solution. Disregard any peak occurring in the test solution at the same RRT as the Methyl Parabens peak.

### Calculations

A. Impurity 1: lactam {cyclohexanespiro (4,5) decane-2,3-butyrolactam}
Note:
Identify the lactam peak based on the retention time in the chromatograms of the Lactam Maker Solution injection.
Resolve Response Factor for lactam (RRF)=21

$$\% Lactam = \frac{{A}_{T}}{{A}_{S}} \times \frac{1}{21} \times \frac{Ws}{50} \times \frac{6}{50} \times \frac{P}{100} \times \frac{50}{WT} \times \frac{Average Filled Wt .}{Label Claim} \times 100$$

### Where,

AT=Peak area of lactam in Test Solution
AS=Peak area of Gabapentin in Standard Solution
P=Potency of Gabapentin W.S.
WS=Weight of Gabapentin W.S. in mg
WT=Weight of Test sample in mg.

B. Single Largest Individual Unidentified Impurities/Degradant
Determine the peak areas for individual impurities/degradant. For the largest peak areas observed other than those of diluent, lactam and Gabapentin peaks

$$\% Single largest individual impurities / degradants$$

$$\frac{AT}{AS} \times \frac{Ws}{50} \times \frac{6}{50} \times \frac{P}{100} \times \frac{50}{WT} \times \frac{Average Filled Wt .}{Label Claim} \times 100$$

### Where,

AT=Peak area of any impurity in Test Solution
AS=Peak area of Gabapentin in Standard Solution
P=Potency of Gabapentin W.S.
WS=Weight of Gabapentin W.S. in mg.
WT=Weight of Test sample in mg.

C. Total other impurities/degradants:

Sum the peak areas of all unidentified impurities.

$$\% Total other impurities / degradants = \frac{\sum AT}{AS} \times \frac{Ws}{50} \times \frac{6}{50} \times \frac{P}{100} \times \frac{50}{WT} \times \frac{Average Filled Wt .}{Label Claim} \times 100$$

### Where,

ΣAT=Peak area of any impurity in Test Solution
AS=Peak area of Gabapentin in Standard Solution
P=Potency of Gabapentin W.S.
WS=Weight of Gabapentin W.S. in mg.
WT=Weight of Test sample in mg.

D. % Total Impurities/degradant:

=% lactam+% total other impurities/degradants

### Medicinal Products

Exemplary medicinal products containing gabapentin are disclosed in Table 1. Table 1 relates to gabapentin formulations containing active doses at 100, 200 and 400 mg. In hard gelatine capsules. Excipients include microcrystalline cellulose as the sole diluent and magnesium stearate as a lubricant. Table 1 additionally sets out capsule shell constituents and also constituents of the printing ink.

Stability data for the formulations of Table 1 at a range of temperatures (20° C. to 40° C.) and durations is provided in Tables 4 through 18.

### Composition

Composition of Proprietary Medicinal Product

TABLE 1
Composition of the proprietary medical product

| | mg/unit | | | | Reference |
|---|---|---|---|---|---|
| Name of Ingredients | 100 mg | 300 mg | 400 mg | Function | to standards |
| Active Ingredient | | | | | |
| Gabapentin | 100.00 | 300.00 | 400.00 | Active | HSE |
| Other Ingredients | | | | | |
| Cellulose, microcrystalline (Avicel | 11.75 | 35.25 | 47.00 | Diluent | Ph. Eur. |
| PH 200) | | | | | |
| Magnesium stearate | 1.50 | 4.50 | 6.00 | Lubricant | Ph. Eur. |
| Total fill weight | 113.25 | 339.75 | 453.00 | | |
| Empty Hard Gelatin Capsule | Size ‘3’ | Size ‘1’ | Size ‘0’ | Capsule shell | HSE |
| Shell | | | | | |
| Methyl parahydroxybenzoate | 0.400 | 0.620 | 0.784 | | Ph. Eur. |
| (E218) | | | | | |
| Propyl parahydroxybenzoate | 0.100 | 0.155 | 0.196 | | Ph. Eur. |
| (E216) | | | | | |
| Sodium laurilsulfate | 0.040 | 0.062 | 0.078 | | Ph. Eur. |
| Titanium oxide (E171) | 1.083 | 0.839 | 1.304 | | Ph. Eur. |
| Yellow iron oxide (E172) | — | 0.465 | 0.784 | | HSE |
| Red iron oxide (E172) | — | — | 0.078 | | HSE |
| Purified water | 7.250 | 11.238 | 14.210 | | Ph. Eur. |
| Gelatin | 41.127 | 64.121 | 80.554 | | Ph. Eur. |
| Constituents of the printing ink | | | | | |
| Ethanol anhydrous | | | | | Ph. Eur. |
| Isopropyl alcohol | | | | | Ph. Eur. |
| Shellac | | | | | Ph. Eur. |
| Activated charcoal | | | | | Ph. Eur. |

### Stability

Stability Tests on the Finished Product

Quality Specification for the proposed shelf-life

The Stability specification for Gabapentin Capsules 100 mg, 300 mg and 400 mg is presented in Table 2

TABLE 2
Stability Specification and routine tests for Gabapentin Capsules

| | Specification | | |
|---|---|---|---|
| Test | 100 mg capsule | 300 mg capsule | 400 mg capsule |
| Appearance (Visual)* | White/white Size ‘3’ hard | Yellow/yellow Size ‘1’ hard | Orange/orange Size ‘0’ hard |
| | gelatin capsules containing | gelatin capsules containing | gelatin capsules containing |
| | white to off white powder | white to off white powder | white to off white powder |
| | printed ‘GAB 100’ and twin | printed with ‘GAB 300’ and | printed with ‘GAB 400’ and |
| | triangle logo in black ink | twin triangle logo in black ink | twin triangle logo in black ink |
| Average capsule weight | 163.2 mg ± 5% | 415.7 mg ± 5% | 548.0 mg ± 5% |
| Average filled weight | 113.2 mg ± 5% | 339.7 mg ± 5% | 453.0 mg ± 5% |
| Uniformity of filled | ±10% of average filled | ±7.5% of average filled | ±7.5% of average filled |
| weight | weight | weight | weight |
| Disintegration (Ph. Eur.) | NMT 15 minutes | NMT 15 minutes | NMT 15 minutes |
| Water content (by KF) | NMT 3% | NMT 3% | NMT 3% |
| Related Substances | | | |
| (TA 02) | | | |
| Lactam | NMT 0.3% | NMT 0.3% | NMT 0.3% |
| Any other impurities | NMT 0.1% | NMT 0.1% | NMT 0.1% |
| Total Impurities | NMT 1.0% | NMT 1.0% | NMT 1.0% |
| (including Lactam) | | | |
| Dissolution (TA 03) | NLT 80% in 20 minutes | NLT 80% in 20 minutes | NLT 80% in 20 minutes |
| Assay: Content of | 95.0-105.0% | 95.0-105.0% | 95.0-105.0% |
| Gabapentin (TA 05) | | | |
| Microbial Limits⁽¹⁾ | NMT 1000 bacteria per gm | NMT 1000 bacteria per gm | NMT 1000 bacteria per gm |
| | NMT 100 fungi per gm. | NMT 100 fungi per gm. | NMT 100 fungi per gm. |
| | E. coli - absent | E. coli - absent | E. coli - absent |

⁽¹⁾To be tested on initial, 6, 24 and 36 months.

### Batches Tested and Packaging

TABLE 3
Details of batches put on stability

| | | Drug | Batch | | |
|---|---|---|---|---|---|
| Capsule | | substance | size | | Date on |
| strength | Batch | batch | (Cap- | Date of | stability |
| (mg) | number | number | sules) | manufacture | test |
| 100 mg | GBC-I/100 | 28800398 | 100,000 | April 1999 | July 1999 |
| | | 288010498 | | | |
| 100 mg | GBC-II/100 | 288010498 | 100,000 | April 1999 | July 1999 |
| | | 288070399 | | | |
| 300 mg | GBC-I/300 | 28800398 | 100,000 | March 1999 | May 1999 |
| | | 288010498 | | | |
| 300 mg | GBC-II/300 | 288010498 | 100,000 | April 1999 | May 1999 |
| | | 288070399 | | | |
| 400 mg | GBC-I/400 | 28800398 | 110,000 | March 1999 | May 1999 |
| | | 288010498 | | | |
| 400 mg | GBC-II/400 | 288010498 | 110,000 | April 1999 | May 1999 |
| | | 288070399 | | | |

Active drug substance used for the manufacture of the above batches was supplied from Teva. All batches were manufactured at Nicholas Piramal (Pithampur) Limited, India.

The above stability batches were packed into white opaque PVC/PVdC/Aluminium blister strips. These blister strips were cartooned prior to being placed on test.

### Storage Conditions

### Real Time Studies

Stability samples were stored at 25° C.±2° C./60%±5% RH and 30° C.±2° C./60%±5% RH and were tested at initial, 3, 6, 12, 18 and 24 and 36 month time points and 3, 6, 12, 18 and 24 month time points respectively.

### Studies Under Other Conditions (Accelerated Conditions)

Stability samples were stored at 40° C.±2° C./75%±5% RH and were tested at initial, 1 month, 2 months, 3 months and 6 months time points.

### Evaluation Test Procedures

The analytical methods for all the tests used in the stability studies are the same as proposed for routine batch analysis and are known to persons skilled in the art. The methodology for, related substances and assay has been validated and are suitable for stability purposes.

The assay and related substances methods used throughout the stability studies are stability indicating.

### Results of Tests

### Results of Physical Testing

The stability data is for 6 months for all strengths at accelerated conditions and for 36 months at 25° C.±2° C./60%±5% RH and 24 months at 30° C.±2° C./60%±5% RH. The results of physical testing of the stability batches packed in PVC/PVdC/Aluminium blister packs is shown in Tables 4 to 19.

Throughout the period of study under all the conditions 25° C.±2° C./60%5% RH, 30° C.±2° C./60%±5% RH and 40° C.±2° C./75%±5% RH, no significant changes were noted in the appearance or disintegration time of any of the samples on test. It is noted none of the stability batches have the markings proposed for marketing, however this does not affect the stability profile.

The percentage water content by KF had shown an increase after one month study in the test samples for 300 and 400 mg strengths.

However, at the end of the second month, once again a similar trend was observed and investigation was initiated as per the SOP for out of specification. The result of the investigation indicated that the test was performed after 6-7 hours of removal of the powder blend from the capsule. The exposure to atmosphere could have resulted in higher values. The statement to the effect that KF should be done on fresh samples only has been included in the method of analysis.

### Results of Chemical Testing

### Related Substances/Impurities

The amount of all the secondary peaks obtained was calculated with respect to Gabapentin diluted standard. In the determination of the amount of known impurity i.e. lactam, the higher response of this impurity (RRF=21 relative to gabapentin) was accounted for in the calculation. From Tables 4 to 19, it may be noted that the value for lactam is well below 0.2% up to 3 months interval for all the strengths. However, at the sixth month interval, the values obtained were slightly above 0.2%.

One unknown impurity at a RRT of about 6.0 was noted under accelerated conditions (40° C.±2° C./75%±5% RH) at the end of one month. Investigation was taken up with respect to the identification and characterisation of this impurity and it was found to be due to the preservative, methyl parabens, present in the capsule shells. The methodology was therefore revised to include preparation of a methyl parabens marker solution and to disregard any peaks occurring in the test samples at the same retention time as the marker.

Total impurities were found to be within the shelf life limits proposed.

### Dissolution

No significant changes were observed in the dissolution results of any of the samples under test.

### Assay

Up to 36 months data at 25° C.2±° C./60%±5% RH, 24 months data at 30° C.±2° C./60%±5% RH and 6 months at 40° C.±2° C./75%±5% RH are available for all strengths of capsules. The data are within specification limits for all the batches.

TABLE 4
Stability of Gabapentin Capsules 100 mg stored in PVC/PVdC/Aluminium packs
(Batch Number GBC-I/100) stored at 25° C. ± 2° C./60% ± 5% RH

| | | | 1 | 2 | 3 | 6 | 9 | 12 | 18 | |
|---|---|---|---|---|---|---|---|---|---|---|
| Test Performed | Limits | Initial | Month | Month | Month | Month | Month | Month | Month | 24 Month |
| Appearance | White/white, Size ‘3’ | White/white, Size ‘3’ | NT | NT | As | As | As | As | As | As initial |
| | hard gelatin capsules | hard gelatin capsules | | | initial | initial | initial | initial | initial | |
| | containing white to off | containing white to off | | | | | | | | |
| | white powder, printed | white powder, printed | | | | | | | | |
| | with logo in black ink. | with logo in black ink. | | | | | | | | |
| Disintegration time | NMT 15 minutes | 8-9 | NT | NT | 8-9 | 8-9 | 6-7 | 7-8 | 6-7 | 6-7 |
| Water content (%) | NMT 3% | 1.65 | NT | NT | 1.75 | 1.82 | 1.75 | 1.70 | 1.51 | 1.56 |
| Related Substances | | | | | | | | | | |
| Lactam | NMT 0.3% | NIL | NT | NT | 0.040 | 0.069 | 0.136 | 0.146 | 0.208 | 0.157 |
| Any other individual | NMT 0.1% | <0.001 | NT | NT | 0.002 | 0.003 | 0.004 | 0.004 | 0.007 | 0.007 |
| impurities | | | | | | | | | | |
| Total Impurities | NMT 1.0% | <0.001 | NT | NT | 0.085 | 0.120 | 0.256 | 0.277 | 0.410 | 0.269 |
| Dissolution | NLT 80% dissolved in | 99.80 | NT | NT | 100.3 | 99.37 | 98.54 | 97.95 | 100.00 | 98.99 |
| | 20 minutes | | | | | | | | | |
| Assay | 95-105% | 98.20 | NT | NT | 98.53 | 98.78 | 98.79 | 98.36 | 98.56 | 98.62 |
| Microbial Limits | NMT 1000 bacteria per | 10 CFU/gm. | NT | NT | NT | NT | NT | NT | NT | NT |
| | gm | | | | | | | | | |
| | NMT 100 fungi per gm. | E. coli - absent | | | | | | | | |
| | E. coli - absent | | | | | | | | | |

NT: Not Tested

TABLE 5
Stability of Gabapentin Capsules 100 mg stored in PVC/PVdC/Aluminium packs
(Batch Number GBC-I/100) stored at 30° C. ± 2° C./60% ± 5% RH

| | | 1 | | 2 | 3 | 6 | 9 | 12 | 18 | |
|---|---|---|---|---|---|---|---|---|---|---|
| Test Performed | Limits | Initial | Month | Month | Month | Month | Month | Month | Month | 24 Month |
| Appearance | White/white, Size ‘3’ | White/white, Size ‘3’ | NT | NT | As | As | As | As | As | As initial |
| | hard gelatin capsules | hard gelatin capsules | | | initial | initial | initial | initial | initial | |
| | containing white to off | containing white to off | | | | | | | | |
| | white powder, printed | white powder, printed | | | | | | | | |
| | with logo in black ink. | with logo in black ink. | | | | | | | | |
| Disintegration time | NMT 15 minutes | 8-9 | NT | NT | 6-7 | 7-8 | 7-8 | 8-9 | 8-9 | 8-9 |
| Water content (%) | NMT 3% | 1.65 | NT | NT | 1.70 | 1.80 | 1.78 | 1.69 | 1.69 | 1.49 |
| Related Substances | | | | | | | | | | |
| Lactam | NMT 0.3% | NIL | NT | NT | 0.062 | 0.096 | 0.136 | 0.223 | 0.260 | 0.267 |
| Any other individual | NMT 0.1% | <0.001 | NT | NT | 0.002 | 0.003 | 0.004 | 0.005 | 0.008 | 0.009 |
| impurities | | | | | | | | | | |
| Total Impurities | NMT 1.0% | <0.001 | NT | NT | 0.099 | 0.250 | 0.252 | 0.415 | 0.465 | 0.363 |
| Dissolution | NLT 80% dissolved in | 99.80 | NT | NT | 87.22 | 99.29 | 98.92 | 99.70 | 99.80 | 99.02 |
| | 20 minutes | | | | | | | | | |
| Assay | 95-105% | 98.20 | NT | NT | 98.32 | 98.41 | 98.93 | 98.92 | 98.38 | 98.45 |
| Microbial Limits | NMT 1000 bacteria per | 10 CFU/gm. | NT | NT | NT | NT | NT | NT | NT | NT |
| | gm | | | | | | | | | |
| | NMT 100 fungi per gm. | E. coli - absent | | | | | | | | |
| | E. coli - absent | | | | | | | | | |

NT: Not Tested

TABLE 6
Stability of Gabapentin Capsules 100 mg stored in PVC/PVdC/Aluminium packs
(Batch Number GBC-I/100) stored at 40° C. ± 2° C./75% ± 5% RH

| Test Performed | Limits | Initial | 1 Month | 2 Month | 3 Month | 6 Month |
|---|---|---|---|---|---|---|
| Appearance | White/white, Size ‘3’ | White/white, Size ‘3’ | As initial | As initial | As initial | As initial |
| | hard gelatin capsules | hard gelatin capsules | | | | |
| | containing white to off | containing white to off | | | | |
| | white powder, printed | white powder, printed | | | | |
| | with logo in black ink. | with logo in black ink. | | | | |
| Disintegration time | NMT 15 minutes | 8-9 | 8-9 | 8-9 | 8-9 | 7-8 |
| Water content (%) | NMT 3% | 1.65 | 1.65 | 1.82 | 1.84 | 1.82 |
| Related Substances | | | | | | |
| Lactam | NMT 0.3% | NIL | 0.064 | 0.145 | 0.144 | 0.206 |
| Any other individual | NMT 0.1% | <0.001 | 0.017 | 0.046 | 0.061 | 0.079 |
| impurities | | | | | | |
| Total Impurities | NMT 1.0% | <0.001 | 0.160 | 0.334 | 0.375 | 0.585 |
| Dissolution | NLT 80% dissolved in | 99.80 | 98.96 | 97.66 | 102.6 | 99.53 |
| | 20 minutes | | | | | |
| Assay | 95-105% | 98.20 | 98.46 | 98.32 | 98.82 | 98.94 |
| Microbial Limits | NMT 1000 bacteria per | 10 CFU/gm. | NT | NT | NT | NT |
| | gm | | | | | |
| | NMT 100 fungi per gm. | E. coli - absent | | | | |
| | E. coli - absent | | | | | |

NT: Not Tested

TABLE 7
Stability of Gabapentin Capsules 100 mg stored in PVC/PVdC/Aluminium packs
(Batch Number GBC-II/100) stored at 25° C. ± 2° C./60% ± 5% RH

| | | | 1 | 2 | 3 | 6 | 9 | 12 | 18 | |
|---|---|---|---|---|---|---|---|---|---|---|
| Test Performed | Limits | Initial | Month | Month | Month | Month | Month | Month | Month | 24 Month |
| Appearance | White/white, Size ‘3’ | White/white, Size ‘3’ | NT | NT | As | As | As | As | As | As initial |
| | hard gelatin capsules | hard gelatin capsules | | | initial | initial | initial | initial | initial | |
| | containing white to off | containing white to off | | | | | | | | |
| | white powder, printed | white powder, printed | | | | | | | | |
| | with logo in black ink. | with logo in black ink. | | | | | | | | |
| Disintegration time | NMT 15 minutes | 6-7 | NT | NT | 6-7 | 8-9 | 6-7 | 8-9 | 7-8 | 8-9 |
| Water content (%) | NMT 3% | 1.68 | NT | NT | 1.85 | 1.71 | 1.70 | 1.65 | 1.53 | 1.58 |
| Related Substances | | | | | | | | | | |
| Lactam | NMT 0.3% | NIL | NT | NT | 0.021 | 0.046 | 0.097 | 0.138 | 0.168 | 0.155 |
| Any other individual | NMT 0.1% | <0.001 | NT | NT | 0.002 | 0.003 | 0.004 | 0.004 | 0.008 | 0.007 |
| impurities | | | | | | | | | | |
| Total Impurities | NMT 1.0% | <0.021 | NT | NT | 0.045 | 0.113 | 0.214 | 0.302 | 0.277 | 0.278 |
| Dissolution | NLT 80% dissolved in | 102.70 | NT | NT | 106.45 | 98.85 | 99.86 | 100.37 | 100.03 | 98.94 |
| | 20 minutes | | | | | | | | | |
| Assay | 95-105% | 98.10 | NT | NT | 99.58 | 98.91 | 98.68 | 98.65 | 98.67 | 98.52 |
| Microbial Limits | NMT 1000 bacteria per | 10 CFU/gm. | NT | NT | NT | NT | NT | NT | NT | NT |
| | gm | | | | | | | | | |
| | NMT 100 fungi per gm. | E. coli - absent | | | | | | | | |
| | E. coli - absent | | | | | | | | | |

NT: Not Tested

TABLE 8
Stability of Gabapentin Capsules 100 mg stored in PVC/PVdC/Aluminium packs
(Batch Number GBC-II/100) stored at 30° C. ± 2° C./60% ± 5% RH

| | | | 1 | 2 | 3 | 6 | 9 | 12 | 18 | 24 |
|---|---|---|---|---|---|---|---|---|---|---|
| Test Performed | Limits | Initial | Month | Month | Month | Month | Month | Month | Month | Month |
| Appearance | White/white, | White/white, | NT | NT | As | As initial | As initial | As initial | As initial | As initial |
| | Size ‘3’ hard | Size ‘3’ hard | | | initial | | | | | |
| | gelatin capsules | gelatin capsules | | | | | | | | |
| | containing white | containing white | | | | | | | | |
| | to off white | to off white | | | | | | | | |
| | powder, printed | powder, printed | | | | | | | | |
| | with logo | with logo | | | | | | | | |
| | in black ink. | in black ink. | | | | | | | | |
| Disintegration time | NMT 15 minutes | 6-7 | NT | NT | 7-8 | 6-7 | 6-7 | 8-9 | 8-9 | 8-9 |
| Water content (%) | NMT 3% | 1.68 | NT | NT | 1.81 | 1.78 | 1.74 | 1.70 | 1.57 | 1.67 |
| Related Substances | | | | | | | | | | |
| Lactam | NMT 0.3% | NIL | NT | NT | 0.030 | 0.073 | 0.098 | 0.208 | 0.218 | 0.268 |
| Any other individual | NMT 0.1% | <0.001 | NT | NT | 0.001 | 0.003 | 0.004 | 0.005 | 0.007 | 0.010 |
| impurities | | | | | | | | | | |
| Total Impurities | NMT 1.0% | <0.021 | NT | NT | 0.037 | 0.094 | 0.213 | 0.381 | 0.367 | 0.344 |
| Dissolution | NLT 80% dissolved | 102.70 | NT | NT | 109.37 | 99.01 | 101.90 | 99.59 | 99.79 | 99.22 |
| | in 20 minutes | | | | | | | | | |
| Assay | 95-105% | 98.10 | NT | NT | 98.93 | 98.28 | 98.57 | 98.53 | 98.94 | 98.87 |
| Microbial Limits | NMT 1000 bacteria | 10 CFU/gm. | NT | NT | NT | NT | NT | NT | NT | NT |
| | per gm | | | | | | | | | |
| | NMT 100 fungi | E. coli - absent | | | | | | | | |
| | per gm. | | | | | | | | | |
| | E. coli - absent | | | | | | | | | |

NT: Not Tested

TABLE 9
Stability of Gabapentin Capsules 100 mg stored in PVC/PVdC/Aluminium packs
(Batch Number GBC-II/100) stored at 40° C. ± 2° C./75% ± 5% RH

| Test Performed | Limits | Initial | 1 Month | 2 Month | 3 Month | 6 Month |
|---|---|---|---|---|---|---|
| Appearance | White/white, Size ‘3’ | White/white, Size ‘3’ | As initial | As initial | As initial | As initial |
| | hard gelatin capsules | hard gelatin capsules | | | | |
| | containing white to off | containing white to off | | | | |
| | white powder, printed | white powder, printed | | | | |
| | with logo in black ink. | with logo in black ink. | | | | |
| Disintegration time | NMT 15 minutes | 6-7 | 7-8 | 6-7 | 7-8 | 7-8 |
| Water content (%) | NMT 3% | 1.68 | 1.66 | 1.87 | 1.80 | 1.82 |
| Related Substances | | | | | | |
| Lactam | NMT 0.3% | NIL | 0.039 | 0.117 | 0.103 | 0.184 |
| Any other individual | NMT 0.1% | <0.001 | 0.017 | 0.043 | 0.054 | 0.073 |
| impurities | | | | | | |
| Total Impurities | NMT 1.0% | <0.021 | 0.221 | 0.239 | 0.384 | 0.579 |
| Dissolution | NLT 80% dissolved in | 102.70 | 103.84 | 103.8 | 108.80 | 98.80 |
| | 20 minutes | | | | | |
| Assay | 95-105% | 98.10 | 100.02 | 99.22 | 98.84 | 98.73 |
| Microbial Limits | NMT 1000 bacteria per | 10 CFU/gm. | NT | NT | NT | NT |
| | gm | | | | | |
| | NMT 100 fungi per gm. | E. coli - absent | | | | |
| | E. coli - absent | | | | | |

NT: Not Tested

TABLE 10
Stability of Gabapentin Capsules 300 mg stored in PVC/PVdC/Aluminium packs
(Batch Number GBC-I/300) stored at 25° C. ± 2° C./60% ± 5% RH

| | | | 1 | 2 | 3 | 6 | 9 | 12 | 18 | 24 |
|---|---|---|---|---|---|---|---|---|---|---|
| Test Performed | Limits | Initial | Month | Month | Month | Month | Month | Month | Month | Month |
| Appearance | Yellow/yellow, | Yellow/yellow, | NT | NT | As initial | As initial | As initial | As initial | As initial | As initial |
| | Size ‘1’ hard | Size ‘1’ hard | | | | | | | | |
| | gelatin capsules | gelatin capsules | | | | | | | | |
| | containing white | containing white | | | | | | | | |
| | to off white | to off white | | | | | | | | |
| | powder, printed | powder, printed | | | | | | | | |
| | with logo in | with logo in | | | | | | | | |
| | black ink. | black ink. | | | | | | | | |
| Disintegration time | NMT 15 minutes | 9-10 | NT | NT | 9-10 | 8-9 | 7-8 | 7-8 | 6-7 | 7-8 |
| Water content (%) | NMT 3% | 1.19 | NT | NT | 1.22 | 1.17 | 1.12 | 1.12 | 1.04 | 0.90 |
| Related Substances | | | | | | | | | | |
| Lactam | NMT 0.3% | NIL | NT | NT | 0.016 | 0.032 | 0.050 | 0.061 | 0.099 | 0.134 |
| Any other individual | NMT 0.1% | <0.001 | NT | NT | <0.001 | <0.001 | 0.001 | 0.001 | 0.001 | 0.002 |
| impurities | | | | | | | | | | |
| Total Impurities | NMT 1.0% | 0.011 | NT | NT | <0.019 | <0.088 | 0.109 | 0.154 | 0.293 | 0.279 |
| Dissolution | NLT 80% | 102.80 | NT | NT | 104.62 | 99.63 | 99.01 | 96.14 | 99.59 | 98.72 |
| | dissolved in | | | | | | | | | |
| | 20 minutes | | | | | | | | | |
| Assay | 95-105% | 100.40 | NT | NT | 100.54 | 98.56 | 98.60 | 99.10 | 99.01 | 101.06 |
| Microbial Limits | NMT 1000 | 10 CFU/gm. | NT | NT | NT | NT | NT | NT | NT | NT |
| | bacteria per gm | | | | | | | | | |
| | NMT 100 fungi | E. coli - absent | | | | | | | | |
| | per gm. | | | | | | | | | |
| | E. coli - absent | | | | | | | | | |

NT: Not Tested

TABLE 11
Stability of Gabapentin Capsules 300 mg stored in PVC/PVdC/Aluminium packs
(Batch Number GBC-I/300) stored at 30° C. ± 2° C./60% ± 5% RH

| | | | 1 | 2 | | | | | | |
|---|---|---|---|---|---|---|---|---|---|---|
| Test Performed | Limits | Initial | Month | Month | 3 Month | 6 Month | 9 Month | 12 Month | 18 Month | 24 Month |
| Appearance | Yellow/yellow, | Yellow/yellow, | NT | NT | As initial | As initial | As initial | As initial | As initial | As initial |
| | Size ‘1’ hard | Size ‘1’ hard | | | | | | | | |
| | gelatin capsules | gelatin capsules | | | | | | | | |
| | containing white | containing white | | | | | | | | |
| | to off white | to off white | | | | | | | | |
| | powder, printed | powder, printed | | | | | | | | |
| | with logo | with logo | | | | | | | | |
| | in black ink. | in black ink. | | | | | | | | |
| Disintegration time | NMT 15 minutes | 9-10 | NT | NT | 8-9 | 9-10 | 8-9 | 8-9 | 8-9 | 6-7 |
| Water content (%) | NMT 3% | 1.19 | NT | NT | 1.20 | 1.13 | 1.14 | 1.12 | 1.02 | 0.89 |
| Related Substances | | | | | | | | | | |
| Lactam | NMT 0.3% | NIL | NT | NT | 0.017 | 0.043 | 0.067 | 0.122 | 0.176 | 0.209 |
| Any other individual | NMT 0.1% | <0.001 | NT | NT | <0.001 | <0.001 | 0.001 | 0.006 | 0.002 | 0.003 |
| impurities | | | | | | | | | | |
| Total Impurities | NMT 1.0% | 0.011 | NT | NT | <0.079 | <0.093 | 0.394 | 0.328 | 0.465 | 0.471 |
| Dissolution | NLT 80% | 102.80 | NT | NT | 101.6 | 99.27 | 101.70 | 98.14 | 98.37 | 99.59 |
| | dissolved in | | | | | | | | | |
| | 20 minutes | | | | | | | | | |
| Assay | 95-105% | 100.40 | NT | NT | 100.26 | 99.01 | 99.01 | 99.10 | 98.92 | 100.54 |
| Microbial Limits | NMT 1000 | 10 CFU/gm. | NT | NT | NT | NT | NT | NT | NT | NT |
| | bacteria per gm | | | | | | | | | |
| | NMT 100 fungi | E. coli - absent | | | | | | | | |
| | per gm. | | | | | | | | | |
| | E. coli - absent | | | | | | | | | |

NT: Not Tested

TABLE 12
Stability of Gabapentin Capsules 300 mg stored in PVC/PVdC/Aluminium packs
(Batch Number GBC-II/300) stored at 30° C. ± 2° C./60% ± 5% RH

| | | | 1 | 2 | | | | | | |
|---|---|---|---|---|---|---|---|---|---|---|
| Test Performed | Limits | Initial | Month | Month | 3 Month | 6 Month | 9 Month | 12 Month | 18 Month | 24 Month |
| Appearance | Yellow/yellow, | Yellow/yellow, | NT | NT | As initial | As initial | As initial | As initial | As initial | As initial |
| | Size ‘1’ hard | Size ‘1’ hard | | | | | | | | |
| | gelatin capsules | gelatin capsules | | | | | | | | |
| | containing white | containing white | | | | | | | | |
| | to off white | to off white | | | | | | | | |
| | powder, printed | powder, printed | | | | | | | | |
| | with logo | with logo | | | | | | | | |
| | in black ink. | in black ink. | | | | | | | | |
| Disintegration time | NMT 15 minutes | 6-7 | NT | NT | 8-9 | 7-8 | 8-9 | 7-8 | 7-8 | 7-8 |
| Water content (%) | NMT 3% | 0.54 | NT | NT | 1.21 | 1.16 | 1.12 | 1.15 | 1.00 | 0.96 |
| Related Substances | | | | | | | | | | |
| Lactam | NMT 0.3% | NIL | NT | NT | 0.016 | 0.037 | 0.055 | 0.106 | 0.144 | 0.189 |
| Any other individual | NMT 0.1% | <0.001 | NT | NT | <0.001 | 0.002 | 0.002 | 0.003 | 0.002 | 0.003 |
| impurities | | | | | | | | | | |
| Total Impurities | NMT 1.0% | <0.012 | NT | NT | <0.177 | 0.234 | 0.219 | 0.209 | 0.317 | 0.459 |
| Dissolution | NLT 80% | 98.06 | NT | NT | 100.86 | 95.70 | 101.38 | 102.60 | 102.14 | 101.92 |
| | dissolved in | | | | | | | | | |
| | 20 minutes | | | | | | | | | |
| Assay | 95-105% | 99.60 | NT | NT | 98.05 | 98.04 | 98.90 | 99.04 | 98.71 | 98.37 |
| Microbial Limits | NMT 1000 | 10 CFU/gm. | NT | NT | NT | NT | NT | NT | NT | NT |
| | bacteria per gm | | | | | | | | | |
| | NMT 100 fungi | E. coli - absent | | | | | | | | |
| | per gm. | | | | | | | | | |
| | E. coli - absent | | | | | | | | | |

NT: Not Tested

TABLE 13
Stability of Gabapentin Capsules 300 mg stored in PVC/PVdC/Aluminium packs
(Batch Number GBC-II/300) stored at 25° C. ± 2° C./60% ± 5% RH

| | | | 1 | 2 | | | | | | |
|---|---|---|---|---|---|---|---|---|---|---|
| Test Performed | Limits | Initial | Month | Month | 3 Month | 6 Month | 9 Month | 12 Month | 18 Month | 24 Month |
| Appearance | Yellow/yellow, | Yellow/yellow, | NT | NT | As initial | As initial | As initial | As initial | As initial | As initial |
| | Size ‘1’ hard | Size ‘1’ hard | | | | | | | | |
| | gelatin capsules | gelatin capsules | | | | | | | | |
| | containing white | containing white | | | | | | | | |
| | to off white | to off white | | | | | | | | |
| | powder, printed | powder, printed | | | | | | | | |
| | with logo | with logo | | | | | | | | |
| | in black ink. | in black ink. | | | | | | | | |
| Disintegration time | NMT 15 minutes | 6-7 | NT | NT | 7-8 | 7-8 | 7-8 | 6-7 | 7-8 | 6-7 |
| Water content (%) | NMT 3% | 0.54 | NT | NT | 1.27 | 1.20 | 1.14 | 1.13 | 1.00 | 0.92 |
| Related Substances | | | | | | | | | | |
| Lactam | NMT 0.3% | NIL | NT | NT | 0.011 | 0.028 | 0.042 | 0.060 | 0.066 | 0.111 |
| Any other individual | NMT 0.1% | <0.001 | NT | NT | <0.001 | 0.001 | 0.002 | 0.002 | 0.002 | 0.002 |
| impurities | | | | | | | | | | |
| Total Impurities | NMT 1.0% | <0.012 | NT | NT | <0.126 | 0.158 | 0.108 | 0.233 | 0.260 | 0.263 |
| Dissolution | NLT 80% | 98.06 | NT | NT | 101.49 | 97.74 | 101.66 | 101.32 | 99.88 | 100.60 |
| | dissolved in | | | | | | | | | |
| | 20 minutes | | | | | | | | | |
| Assay | 95-105% | 99.60 | NT | NT | 98.66 | 97.87 | 97.68 | 98.20 | 98.65 | 99.75 |
| Microbial Limits | NMT 1000 | 10 CFU/gm. | NT | NT | NT | NT | NT | NT | NT | NT |
| | bacteria per gm | | | | | | | | | |
| | NMT 100 fungi | E. coli - absent | | | | | | | | |
| | per gm. | | | | | | | | | |
| | E. coli - absent | | | | | | | | | |

NT: Not Tested

TABLE 14
Stability of Gabapentin Capsules 400 mg stored in PVC/PVdC/Aluminium packs
(Batch Number GBC-I/400) stored at 25° C. ± 2° C./60% ± 5% RH

| | | | 1 | 2 | | | | | | |
|---|---|---|---|---|---|---|---|---|---|---|
| Test Performed | Limits | Initial | Month | Month | 3 Month | 6 Month | 9 Month | 12 Month | 18 Month | 24 Month |
| Appearance | Orange/orange, | Orange/orange, | NT | NT | As initial | As initial | As initial | As initial | As initial | As initial |
| | Size ‘1’ hard | Size ‘1’ hard | | | | | | | | |
| | gelatin capsules | gelatin capsules | | | | | | | | |
| | containing white | containing white | | | | | | | | |
| | to off white | to off white | | | | | | | | |
| | powder, printed | powder, printed | | | | | | | | |
| | with logo | with logo | | | | | | | | |
| | in black ink. | in black ink. | | | | | | | | |
| Disintegration time | NMT 15 minutes | 6-7 | NT | NT | 7-8 | 6-7 | 7-8 | 7-8 | 7-8 | 6-7 |
| Water content (%) | NMT 3% | 1.19 | NT | NT | 1.20 | 1.14 | 1.20 | 1.06 | 1.06 | 0.99 |
| Related Substances | | | | | | | | | | |
| Lactam | NMT 0.3% | NIL | NT | NT | 0.013 | 0.025 | 0.042 | 0.058 | 0.074 | 0.104 |
| Any other individual | NMT 0.1% | <0.001 | NT | NT | <0.001 | <0.001 | 0.001 | 0.001 | 0.001 | 0.002 |
| impurities | | | | | | | | | | |
| Total Impurities | NMT 1.0% | <0.001 | NT | NT | <0.099 | <0.050 | 0.187 | 0.221 | 0.212 | 0.196 |
| Dissolution | NLT 80% | 101.70 | NT | NT | 99.25 | 99.20 | 100.37 | 98.45 | 99.64 | 98.92 |
| | dissolved in | | | | | | | | | |
| | 20 minutes | | | | | | | | | |
| Assay | 95-105% | 101.70 | NT | NT | 98.30 | 98.09 | 98.12 | 98.52 | 98.13 | 99.40 |
| Microbial Limits | NMT 1000 | 10 CFU/gm. | NT | NT | NT | NT | NT | NT | NT | NT |
| | bacteria per gm | | | | | | | | | |
| | NMT 100 fungi | E. coli - absent | | | | | | | | |
| | per gm. | | | | | | | | | |
| | E. coli - absent | | | | | | | | | |

NT: Not Tested

TABLE 15
Stability of Gabapentin Capsules 400 mg stored in PVC/PVdC/Aluminium packs
(Batch Number GBC-I/400) stored at 30° C. ± 2° C./60% ± 5% RH

| | | | 1 | 2 | | | | | | |
|---|---|---|---|---|---|---|---|---|---|---|
| Test Performed | Limits | Initial | Month | Month | 3 Month | 6 Month | 9 Month | 12 Month | 18 Month | 24 Month |
| Appearance | Orange/orange, | Orange/orange, | NT | NT | As initial | As initial | As initial | As initial | As initial | As initial |
| | Size ‘1’ hard | Size ‘1’ hard | | | | | | | | |
| | gelatin capsules | gelatin capsules | | | | | | | | |
| | containing white | containing white | | | | | | | | |
| | to off white | to off white | | | | | | | | |
| | powder, printed | powder, printed | | | | | | | | |
| | with logo | with logo | | | | | | | | |
| | in black ink. | in black ink. | | | | | | | | |
| Disintegration time | NMT 15 minutes | 6-7 | NT | NT | 6-7 | 7-8 | 6-7 | 6-7 | 7-8 | 6-7 |
| Water content (%) | NMT 3% | 1.19 | NT | NT | 1.23 | 1.18 | 1.08 | 1.19 | 1.02 | 1.00 |
| Related Substances | | | | | | | | | | |
| Lactam | NMT 0.3% | NIL | NT | NT | 0.016 | 0.037 | 0.05 | 0.090 | 0.125 | 0.184 |
| Any other individual | NMT 0.1% | <0.001 | NT | NT | 0.001 | <0.001 | 0.001 | 0.002 | 0.002 | 0.003 |
| impurities | | | | | | | | | | |
| Total Impurities | NMT 1.0% | <0.001 | NT | NT | 0.234 | <0.174 | 0.171 | 0.243 | 0.343 | 0.410 |
| Dissolution | NLT 80% | 101.70 | NT | NT | 100.25 | 99.20 | 99.42 | 97.33 | 101.28 | 98.21 |
| | dissolved in | | | | | | | | | |
| | 20 minutes | | | | | | | | | |
| Assay | 95-105% | 101.70 | NT | NT | 99.35 | 98.09 | 98.48 | 98.14 | 98.25 | 97.42 |
| Microbial Limits | NMT 1000 | 10 CFU/gm. | NT | NT | NT | NT | NT | NT | NT | NT |
| | bacteria per gm | | | | | | | | | |
| | NMT 100 fungi | E. coli - absent | | | | | | | | |
| | per gm. | | | | | | | | | |
| | E. coli - absent | | | | | | | | | |

NT: Not Tested

TABLE 16
Stability of Gabapentin Capsules 400 mg stored in PVC/PVdC/Aluminium packs
(Batch Number GBC-I/400) stored at 40° C. ± 2° C./75% ± 5% RH

| Test Performed | Limits | Initial | 1 Month | 2 Month | 3 Month | 6 Month |
|---|---|---|---|---|---|---|
| Appearance | Orange/orange, Size ‘0’ | Orange/orange, Size ‘0’ | As initial | As initial | As initial | As initial |
| | hard gelatin capsules | hard gelatin capsules | | | | |
| | containing white to off | containing white to off | | | | |
| | white powder, printed | white powder, printed | | | | |
| | with logo in black ink. | with logo in black ink. | | | | |
| Disintegration time | NMT 15 minutes | 6-7 | 7-8 | 7-8 | 7-8 | 7-8 |
| Water content (%) | NMT 3% | 1.19 | 1.50 | 1.52 | 1.58 | 1.51 |
| Related Substances | | | | | | |
| Lactam | NMT 0.3% | NIL | 0.040 | 0.075 | 0.080 | 0.185 |
| Any other individual | NMT 0.1% | <0.001 | 0.005 | 0.018 | 0.014 | 0.039 |
| impurities | | | | | | |
| Total Impurities | NMT 1.0% | <0.001 | 0.045 | 0.460 | 0.314 | 0.469 |
| Dissolution | NLT 80% dissolved in | 101.70 | 99.64 | 101.54 | 98.93 | 91.59 |
| | 20 minutes | | | | | |
| Assay | 95-105% | 101.70 | 99.28 | 98.52 | 97.84 | 98.23 |
| Microbial Limits | NMT 1000 bacteria per | 10 CFU/gm. | NT | NT | NT | 10 CFU |
| | gm | | | | | absent |
| | NMT 100 fungi per gm. | E. coli - absent | | | | |
| | E. coli - absent | | | | | |

NT: Not Tested

TABLE 17
Stability of Gabapentin Capsules 400 mg stored in PVC/PVdC/Aluminium packs
(Batch Number GBC-II/400) stored at 25° C. ± 2° C./60% ± 5% RH

| | | | 1 | 2 | | | | | | |
|---|---|---|---|---|---|---|---|---|---|---|
| Test Performed | Limits | Initial | Month | Month | 3 Month | 6 Month | 9 Month | 12 Month | 18 Month | 24 Month |
| Appearance | Orange/orange, | Orange/orange, | NT | NT | As initial | As initial | As initial | As initial | As initial | As initial |
| | Size ‘1’ hard | Size ‘1’ hard | | | | | | | | |
| | gelatin capsules | gelatin capsules | | | | | | | | |
| | containing white | containing white | | | | | | | | |
| | to off white | to off white | | | | | | | | |
| | powder, printed | powder, printed | | | | | | | | |
| | with logo | with logo | | | | | | | | |
| | in black ink. | in black ink. | | | | | | | | |
| Disintegration time | NMT 15 minutes | 10-11 | NT | NT | 10-11 | 10-11 | 10-11 | 10-11 | 9-10 | 9-10 |
| Water content (%) | NMT 3% | 0.54 | NT | NT | 1.14 | 1.10 | 1.05 | 1.05 | 1.00 | 0.95 |
| Related Substances | | | | | | | | | | |
| Lactam | NMT 0.3% | NIL | NT | NT | 0.010 | 0.028 | 0.036 | 0.047 | 0.066 | 0.109 |
| Any other individual | NMT 0.1% | NIL | NT | NT | <0.001 | 0.002 | 0.002 | 0.012 | 0.001 | 0.002 |
| impurities | | | | | | | | | | |
| Total Impurities | NMT 1.0% | NIL | NT | NT | <0.035 | 0.030 | 0.059 | 0.219 | 0.186 | 0.195 |
| Dissolution | NLT 80% | 95.30 | NT | NT | 98.23 | 94.06 | 100.72 | 99.47 | 95.80 | 99.24 |
| | dissolved in | | | | | | | | | |
| | 20 minutes | | | | | | | | | |
| Assay | 95-105% | 101.50 | NT | NT | 101.70 | 98.96 | 98.80 | 98.54 | 98.31 | 99.66 |
| Microbial Limits | NMT 1000 | 10 CFU/gm. | NT | NT | NT | NT | NT | NT | NT | NT |
| | bacteria per gm | | | | | | | | | |
| | NMT 100 fungi | E. coli - absent | | | | | | | | |
| | per gm. | | | | | | | | | |
| | E. coli - absent | | | | | | | | | |

NT: Not Tested

TABLE 18
Stability of Gabapentin Capsules 400 mg stored in PVC/PVdC/Aluminium packs
(Batch Number GBC-II/400) stored at 30° C. ± 2° C./60% ± 5% RH

| | | | 1 | 2 | | | | | | |
|---|---|---|---|---|---|---|---|---|---|---|
| Test Performed | Limits | Initial | Month | Month | 3 Month | 6 Month | 9 Month | 12 Month | 18 Month | 24 Month |
| Appearance | Orange/orange, | Orange/orange, | NT | NT | As initial | As initial | As initial | As initial | As initial | As initial |
| | Size ‘1’ hard | Size ‘1’ hard | | | | | | | | |
| | gelatin capsules | gelatin capsules | | | | | | | | |
| | containing white | containing white | | | | | | | | |
| | to off white | to off white | | | | | | | | |
| | powder, printed | powder, printed | | | | | | | | |
| | with logo | with logo | | | | | | | | |
| | in black ink. | in black ink. | | | | | | | | |
| Disintegration time | NMT 15 minutes | 10-11 | NT | NT | 11-12 | 11-12 | 10-11 | 10-11 | 10-11 | 9-10 |
| Water content (%) | NMT 3% | 0.54 | NT | NT | 1.12 | 1.10 | 1.02 | 1.06 | 1.00 | 0.97 |
| Related Substances | | | | | | | | | | |
| Lactam | NMT 0.3% | NIL | NT | NT | 0.013 | 0.036 | 0.052 | 0.072 | 0.131 | 0.185 |
| Any other | NMT 0.1% | NIL | NT | NT | <0.001 | 0.004 | 0.002 | 0.019 | 0.002 | 0.003 |
| individual | | | | | | | | | | |
| impurities | | | | | | | | | | |
| Total | NMT 1.0% | NIL | NT | NT | <0.174 | 0.232 | 0.119 | 0.516 | 0.353 | 0.452 |
| Impurities | | | | | | | | | | |
| Dissolution | NLT 80% | 95.30 | NT | NT | 94.68 | 94.65 | 104.31 | 97.51 | 96.74 | 100.15 |
| | dissolved in | | | | | | | | | |
| | 20 minutes | | | | | | | | | |
| Assay | 95-105% | 101.50 | NT | NT | 101.10 | 99.21 | 99.00 | 98.69 | 98.29 | 98.94 |
| Microbial Limits | NMT 1000 | 10 CFU/gm. | NT | NT | NT | NT | NT | NT | NT | NT |
| | bacteria per gm | | | | | | | | | |
| | NMT 100 fungi | E. coli - absent | | | | | | | | |
| | per gm. | | | | | | | | | |
| | E. coli - absent | | | | | | | | | |

NT: Not Tested

TABLE 19
Stability of Gabapentin Capsules 400 mg stored in PVC/PVdC/Aluminium packs
(Batch Number GBC-II/400) stored at 40° C. ± 2° C./75% ± 5% RH

| Test Performed | Limits | Initial | 1 Month | 2 Month | 3 Month | 6 Month |
|---|---|---|---|---|---|---|
| Appearance | Orange/orange, Size ‘0’ | Orange/orange, Size ‘0’ | As initial | As initial | As initial | As initial |
| | hard gelatin capsules | hard gelatin capsules | | | | |
| | containing white to off | containing white to off | | | | |
| | white powder, printed | white powder, printed | | | | |
| | with logo in black ink. | with logo in black ink. | | | | |
| Disintegration time | NMT 15 minutes | 10-11 | 11-12 | 11-12 | 10-11 | 11-12 |
| Water content (%) | NMT 3% | 0.54 | 1.54 | 1.56 | 1.53 | 1.52 |
| Related Substances | | | | | | |
| Lactam | NMT 0.3% | NIL | 0.044 | 0.083 | 0.104 | 0.219 |
| Any other individual | NMT 0.1% | NIL | 0.007 | 0.024 | 0.023 | 0.042 |
| impurities | | | | | | |
| Total Impurities | NMT 1.0% | NIL | 0.051 | 0.291 | 0.627 | 0.506 |
| Dissolution | NLT 80% dissolved in | 95.30 | 100.48 | 91.85 | 99.79 | 94.10 |
| | 20 minutes | | | | | |
| Assay | 95-105% | 101.50 | 101.09 | 100.38 | 99.80 | 99.12 |
| Microbial Limits | NMT 1000 bacteria per | 10 CFU/gm. | NT | NT | NT | 10 CFU |
| | gm | | | | | absent |
| | NMT 100 fungi per gm. | E. coli - absent | | | | |
| | E. coli - absent | | | | | |

NT: Not Tested

TABLE 20
Stability of Neurontin ® Capsules 400 mg stored in PVC/PVdC/Aluminium packs stored at 25° C. ± 2° C./60% ± 5% RH

| | | | 1 | | | | | | |
|---|---|---|---|---|---|---|---|---|---|
| Test Performed | Limits | Initial | Month | 2 Month | 3 Month | 6 Month | 12 Month | 18 Month | 24 Month |
| Appearance | Orange/orange, Size ‘0’ | Orange/orange, | NT | NT | NT | As initial | As initial | NT | As initial |
| | hard gelatin capsules | Size ‘0’ hard | | | | | | | |
| | containing white to off | gelatin capsules | | | | | | | |
| | white powder, printed | containing white | | | | | | | |
| | with Neurontin ® 400 mg | to off white | | | | | | | |
| | logo in black ink. | powder, printed | | | | | | | |
| | | with Neurontin ® | | | | | | | |
| | | 400 mg | | | | | | | |
| | | logo in black ink. | | | | | | | |
| Disintegration time | NMT 15 minutes | 10-11 | NT | NT | NT | 10-11 | 9-10 | NT | |
| Water content (%) | NMT 3% | NT | NT | NT | NT | 1.38 | 1.36 | NT | |
| Related Substances | | | | | | | | | |
| Lactam | NMT 0.3% | NIL | NT | NT | NT | 0.037 | 0.078 | NT | 0.113 |
| Any other individual | NMT 0.1% | <0.001 | NT | NT | NT | NIL | 0.001 | NT | 0.109 |
| impurities | | | | | | | | | |
| Total Impurities | NMT 1.0% | <0.001 | NT | NT | NT | 0.051 | 0.269 | NT | 0.378 |
| Dissolution | NLT 80% dissolved in | 102.80 | NT | NT | NT | 93.84 | 96.60 | NT | 101.29 |
| | 20 minutes | | | | | | | | |
| Assay | 95-105% | 98.80 | NT | NT | NT | 97.10 | 98.62 | NT | 98.19 |
| Microbial Limits | NMT 1000 bacteria per | NT | NT | NT | NT | NT | NT | NT | NT |
| | gm | | | | | | | | |

NT: Not Tested

TABLE 21
Stability of Neurontin ® Capsules 400 mg stored in PVC/PVdC/Aluminium packs stored at 30° C. ± 2° C./60% ± 5% RH

| | | | 1 | 2 | | | | | | |
|---|---|---|---|---|---|---|---|---|---|---|
| Test Performed | Limits | Initial | Month | Month | 3 Month | 6 Month | 9 Month | 12 Month | 18 Month | 24 Month |
| Appearance | Orange/orange, | Orange/orange, | NT | NT | As initial | As initial | As initial | As initial | As initial | As initial |
| | Size ‘0’ hard | Size ‘0’ hard | | | | | | | | |
| | gelatin capsules | gelatin capsules | | | | | | | | |
| | containing white | containing white | | | | | | | | |
| | to off white | to off white | | | | | | | | |
| | powder, printed | powder, printed | | | | | | | | |
| | with Neurontin ® | with Neurontin ® | | | | | | | | |
| | 400 mg | 400 mg | | | | | | | | |
| | logo in black ink. | logo in black ink. | | | | | | | | |
| Disintegration time | NMT 15 minutes | 10-11 | NT | NT | 10-11 | 10-11 | 9-10 | 9-10 | | |
| Water content (%) | NMT 3% | NT | NT | NT | 1.62 | 1.38 | 1.40 | 1.65 | | |
| Related Substances | | | | | | | | | | |
| Lactam | NMT 0.3% | NIL | NT | NT | 0.016 | 0.049 | 0.071 | 0.118 | 0.152 | 0.190 |
| Any other individual | NMT 0.1% | <0.001 | NT | NT | <0.001 | <0.001 | <0.001 | <0.001 | <0.001 | 0.278 |
| impurities | | | | | | | | | | |
| Total Impurities | NMT 1.0% | <0.001 | NT | NT | <0.072 | <0.111 | 0.134 | 0.329 | <0.374 | 0.824 |
| Dissolution | NLT 80% | 102.80 | NT | NT | 97.80 | 93.84 | 99.13 | 97.78 | 99.23 | 101.76 |
| | dissolved in | | | | | | | | | |
| | 20 minutes | | | | | | | | | |
| Assay | 95-105% | 98.80 | NT | NT | 99.82 | 97.10 | 100.01 | 98.79 | 98.36 | 100.10 |
| Microbial Limits | NMT 1000 | NT | NT | NT | NT | NT | NT | NT | | |
| | bacteria per gm | | | | | | | | | |

NT: Not Tested

TABLE 22
Stability of Neurontin ® Capsules 400 mg stored in PVC/PVdC/Aluminium packs stored at 40° C. ± 2° C./75% ± 5% RH

| Test Performed | Limits | Initial | 1 Month | 2 Month | 3 Month | 6 Month |
|---|---|---|---|---|---|---|
| Appearance | Orange/orange, Size ‘0’ | Orange/orange, Size ‘0’ | As initial | As initial | As initial | As initial |
| | hard gelatin capsules | hard gelatin capsules | | | | |
| | containing white to off | containing white to off | | | | |
| | white powder, printed | white powder, printed | | | | |
| | with Neurontin ® 400 mg | with Neurontin ® 400 mg | | | | |
| | logo in black ink. | logo in black ink. | | | | |
| Disintegration time | NMT 15 minutes | 10-11 | 10-11 | 10-11 | 10-11 | 10-11 |
| Water content (%) | NMT 3% | NT | 1.64 | 1.76 | 1.44 | 1.40 |
| Related Substances | | | | | | |
| Lactam | NMT 0.3% | NIL | <0.077 | 0.103 | 0.135 | 0.283 |
| Any other individual | NMT 0.1% | <0.001 | <0.001 | <0.001 | 0.001 | 0.004 |
| impurities | | | | | | |
| Total Impurities | NMT 1.0% | <0.001 | <0.120 | <0.225 | 0.225 | 1.084 |
| Dissolution | NLT 80% dissolved in | 102.80 | 99.96 | 102.20 | 96.41 | 93.81 |
| | 20 minutes | | | | | |
| Assay | 95-105% | 98.80 | 99.13 | 100.97 | 98.64 | 98.15 |
| Microbial Limits | NMT 1000 bacteria per | NT | NT | NT | NT | NT |
| | gm | | | | | |

NT: Not Tested

### Stability Conclusions

Gabapentin capsules packed into PVC/PVdC/Aluminium blister pack have been shown to be physically and chemically stable for 36 months when stored at 25° C./50%±5% RH, 24 months when stored at 30° C.±2° C./60%±5% H and for 6 months when stored at 40° C.±2° C./75%±5% H.

### Proposed Shelf-life

The stability data generated supports the following:

Proposed product shelf-life: 36 months when packed in blister packs.

Labelled storage conditions: none.

### Further Exemplary Medicinal Products

Further exemplary medicinal products containing the gabapentin active are disclosed in Tables 23 and 24.

Exemplary trial blends for 400 mg formulations are disclosed e.g. in Table 24.

Tables 25 through 39 show stability data for these further exemplary formulations.

### Formulation Development

A capsule formulation was needed which would be linear for all three strengths.

The excipients used in the preformulation studies and the coding are shown in Table 23.

TABLE 23
Excipients used in the pre-formulation studies

| | Sam- | Binary |
|---|---|---|
| | ple | mixture |
| Excipients | code | code |
| DILUENTS | | |
| 1. Dibasic Calcium Phosphate IP (NGRANULES) | D1 | GD1 |
| 2. Tribasic Calcium Phosphate IP | D2 | GD2 |
| 3. Calcium Sulphate anhydrous Ph. Eur | D3 | GD3 |
| 4. Mannitol Ph. Eur | D4 | GD4 |
| 5. Microcrystalline Cellulose (AVICEL PH 200) Ph. Eur | D5 | GD5 |
| 6. Starch IP | D6 | GD6 |
| 7. Lactose (PHARMATOSE) Ph. Eur | D7 | GD7 |
| LUBRICANTS | | |
| 1. Magnesium Stearate Ph. Eur | L1 | GL1 |
| 2. Stearic Acid IP | L2 | GL2 |
| 3. Colloidal Silicon Dioxide Ph. Eur | L3 | GL3 |
| SOLUBILIZER | | |
| 1. Sodium Lauryl Sulphate IP | S1 | GS1 |
| DRUG | | |
| Gabapentin (Recon) HSE | G | |
| Neurontin ® Capsule 400 mg | NRT | |
| B No. 0015077 | | |

Two trial blends (Blend I and Blend II) having the composition as shown in Table 24 were also evaluated as per the protocol for pre-formulation trials.

TABLE 24
Trial blends for pre-formulation studies.

| | Blend I | | Blend II | |
|---|---|---|---|---|
| | per | | per | per 50 |
| | capsule | per 50 capsules | capsule | capsules |
| Ingredients | (mg) | (g) | (mg) | (g) |
| Gabapentin | 400.00 | 20.00 | 400.00 | 20.00 |
| Microcrystalline Cellulose | 133.00 | 6.65 | 133.00 | 6.65 |
| (Avicel PH 200) | | | | |
| Magnesium Stearate | 5.00 | 0.25 | 5.00 | 0.25 |
| Colloidal Silicon Dioxide | 2.00 | 0.10 | — | — |
| Sodium Lauryl Sulphate | 0.20 | 0.01 | 0.20 | 0.01 |

All the ingredients were passed through 20 mesh screen and blended together. The results of all the pre-formulation compatibility studies are given in Tables 25 to 39, including comparative results for the drug substance (Table 39) and UK reference product (Table 38) as controls.

### Excipient Compatibility Study Protocol Gabapentin Capsules

Aim: To carry out preformulation excipient compatibility studies for Gabapentin capsules

### Controls

Gabapentin drug substance

Samples retained at 4° C.

Individual Excipients

### Excipients

### Diluents

Dibasic calcium phosphate

Tribasic calcium phosphate

Calcium sulphate

Mannitol

Microcrystalline cellulose

Starch IP

Lactose

### Lubricants

Magnesium stearate

Steric acid

Colloidal silicon dioxide-to confirm the reported incompatibility

### Solubilizer

Sodium lauryl sulphate

### Drug

Gabapentin

Binary mixtures to be evaluated (with and without water as necessary):

1. Drug and diluents (1:0.5)

2. Drug and lubricants(1:0.1)

3. Drug and solubilizer (1:0.001)

4. Proposed formulation from the in vitro formulation trials to confirm the extent of interactions and identify suitably stable formulations.

5. Other combinations as appropriate

### Scheme to identify chemical compatibility using DSC with confirmatory HPLC.

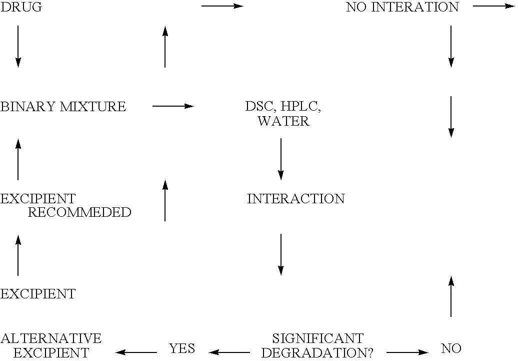

Significant degradation is defined as

1. >0.5% w/w formation of lactam degradation product at conditions up to 40° C./75% RH.

2. Formation of other degradants at levels >0.1% w/w.

3. Greater relative instability of mixture or formulation to Gabapentin drug substance and Neurotonin capsules.

### Storage Conditions

Where humidity controls are not available a defined amount of water may be added to the samples. Storage of samples was in petridishes and in stoppered glass vials.

### Analysis of the Samples

The following tests were performed at each interval:

1. Appearance

2. HPLC assay (as for HPLC related substances assay but calibrated for gabapentin resolution rather than for related substances)

The following tests were performed at 14 and 28 day intervals.

1. Appearance

2. HPLC assay

3. HPLC assay for related substances

4. Water (to determine the hygroscopicity of the proposed formulations.

DSC was carried out on initial and end point stability samples.

### Acceptance Criteria:

Similar stability profile to Neurotonin capsules, similar stability profile to Gabapentin drug substance, lactam levels <0.5% w/w at 25° C. /60% RH and 40° C./75% RH after 28 days, dissolution of the proposed formulation is >80% (Q) in 20 minutes at 25° C. C/60% RH and 40° C./75% RH after 28 days.

TABLE 25
Pre-formulation studies, Batch Number: GD1

| Specification | | | 25° C./60% RH | | | | 40° C./75% RH | |
|---|---|---|---|---|---|---|---|---|
| Test | Requirement | Initial | 3 days | 7 days | 14 days | 28 days | 3 days | 7 days |
| Description | White powder | White | White | White | White | White | White | White |
| | | powder | powder | powder | powder | powder | powder | powder |
| Impurities | | | | | | | | |
| a) Lactam | NMT 0.200% | Nil | — | — | Nil | Nil | — | — |
| content | | | | | | | | |
| b) Single | NMT 0.100% | Nil | — | — | Nil | Nil | — | — |
| largest | | | | | | | | |
| individual | | | | | | | | |
| Impurities | | | | | | | | |
| c) Total | NMT 0.500% | Nil | — | — | Nil | Nil | — | — |
| other | | | | | | | | |
| Impurities | | | | | | | | |
| d) Total | NMT 0.700% | Nil | — | — | Nil | Nil | — | — |
| Impurities | | | | | | | | |
| Assay | 95-105% | 103.4% | 103.3% | 103.5% | 103.2% | 103.2% | 103.1% | 103.2% |
| | | | | | | | | UV |
| | | 40° C./ | | | | | | (254 |
| Specification | | 75% RH | | 50° C. | | | | nm) |
| Test | Requirement | 14 days | 28 days | 3 days | 7 days | 14 days | 28 days | 3 days |
| Description | White powder | White | White | White | White | White | White | White |
| | | powder | powder | powder | powder | powder | powder | pow |
| Impurities | | | | | | | | |
| a) Lactam | NMT 0.200% | Nil | Nil | — | — | Nil | 0.105% | — |
| content | | | | | | | | |
| b) Single | NMT 0.100% | Nil | Nil | — | — | Nil | 0.020% | — |
| largest | | | | | | | | |
| individual | | | | | | | | |
| Impurities | | | | | | | | |
| c) Total | NMT 0.500% | Nil | Nil | — | — | Nil | 0.013% | — |
| other | | | | | | | | |
| Impurities | | | | | | | | |
| d) Total | NMT 0.700% | Nil | Nil | — | — | Nil | 0.138% | — |
| Impurities | | | | | | | | |
| Assay | 95-105% | 103.1% | 102.4% | 103.0% | 103.1% | 102.9% | 102.8% | 103.5% |

— not tested

TABLE 26
Pre-formulation studies, Batch Number: GD2

| Specification | | | 25° C./60% RH | | | | 40° C./75% RH | |
|---|---|---|---|---|---|---|---|---|
| Test | Requirement | Initial | 3 days | 7 days | 14 days | 28 days | 3 days | 7 days |
| Description | White powder | White | White | White | White | White | White | White |
| | | powder | powder | powder | powder | powder | powder | powder |
| Impurities | | | | | | | | |
| a) Lactam | NMT 0.200% | Nil | — | — | Nil | 0.198% | — | — |
| content | | | | | | | | |
| b) Single | NMT 0.100% | 0.020% | — | — | 0.025% | 0.036% | — | — |
| largest | | | | | | | | |
| individual | | | | | | | | |
| Impurities | | | | | | | | |
| c) Total | NMT 0.500% | Nil | — | — | Nil | Nil | — | — |
| other | | | | | | | | |
| Impurities | | | | | | | | |
| d) Total | NMT 0.700% | 0.020% | — | — | 0.025% | 0.234% | — | — |
| Impurities | | | | | | | | |
| Assay | 95-105% | 101.6% | 100.8% | 101.0% | 101.8% | 100.5% | 100.4% | 100.9% |
| Specification | | 40° C./75% RH | | 50° C. | | | | UV |
| Test | Requirement | 14 days | 28 days | 3 days | 7 days | 14 days | 28 days | 3 days |
| Description | White powder | White | White | White | White | White | White | White |
| | | powder | powder | powder | powder | powder | powder | powde |
| Impurities | | | | | | | | |
| a) Lactam | NMT 0.200% | 0.084% | 0.150% | — | — | 0.500% | 0.540% | — |
| content | | | | | | | | |
| b) Single | NMT 0.100% | Nil | Nil | — | — | 0.034% | 0.042% | — |
| largest | | | | | | | | |
| individual | | | | | | | | |
| Impurities | | | | | | | | |
| c) Total | NMT 0.500% | Nil | Nil | — | — | 0.020% | 0.028% | — |
| other | | | | | | | | |
| Impurities | | | | | | | | |
| d) Total | NMT 0.700% | 0.084% | 0.150% | — | — | 0.554% | 0.610% | — |
| Impurities | | | | | | | | |
| Assay | 95-105% | 100.0% | 99.9% | 100.0% | 100.2% | 99.8% | 99.7% | 101.7% |

— not tested

TABLE 27
Pre-formulation studies, Batch Number: GD3

| Specification | | | 25° C./60% RH | | | | 40° C./75% RH | |
|---|---|---|---|---|---|---|---|---|
| Test | Requirement | Initial | 3 days | 7 days | 14 days | 28 days | 3 days | 7 days |
| Description | White powder | White | White | White | White | White | White | White |
| | | powder | powder | powder | powder | powder | powder | powder |
| Impurities | | | | | | | | |
| a) Lactam | NMT 0.200% | Nil | — | — | Nil | Nil | — | — |
| content | | | | | | | | |
| b) Single | NMT 0.100% | Nil | — | — | Nil | 0.008% | — | — |
| largest | | | | | | | | |
| individual | | | | | | | | |
| Impurities | | | | | | | | |
| c) Total | NMT 0.500% | Nil | — | — | Nil | Nil | — | — |
| other | | | | | | | | |
| Impurities | | | | | | | | |
| d) Total | NMT 0.700% | Nil | — | — | Nil | 0.008% | — | — |
| Impurities | | | | | | | | |
| Assay | 95-105% | 104.4% | 104.6% | 104.0% | 104.7% | 104.6% | 104.0% | 103.8% |
| Specification | | 40° C./75% RH | | 50° C. | | | | UV |
| Test | Requirement | 14 days | 28 days | 3 days | 7 days | 14 days | 28 days | 3 days |
| Description | White powder | White | White | White | White | White | White | White |
| | | powder | powder | powder | powder | powder | powder | powder |
| Impurities | | | | | | | | |
| a) Lactam | NMT 0.200% | Nil | Nil | — | — | Nil | Nil | — |
| content | | | | | | | | |
| b) Single | NMT 0.100% | Nil | Nil | — | — | Nil | Nil | — |
| largest | | | | | | | | |
| individual | | | | | | | | |
| Impurities | | | | | | | | |
| c) Total | NMT 0.500% | Nil | Nil | — | — | Nil | Nil | — |
| other | | | | | | | | |
| Impurities | | | | | | | | |
| d) Total | NMT 0.700% | Nil | Nil | — | — | Nil | Nil | — |
| Impurities | | | | | | | | |
| Assay | 95-105% | 103.1% | 103.5% | 103.8% | 103.6% | 102.1% | 102.5% | 104.5% |

— not tested

TABLE 28
Pre-formulation studies, Batch Number: GD4

| Specification | | | 25° C./60% RH | | | | 40° C./75% RH | |
|---|---|---|---|---|---|---|---|---|
| Test | Requirement | Initial | 3 days | 7 days | 14 days | 28 days | 3 days | 7 days |
| Description | White powder | White | White | White | White | White | White | White |
| | | powder | powder | powder | powder | powder | powder | powder |
| Impurities | | | | | | | | |
| a) Lactam | NMT 0.200% | Nil | — | — | Nil | Nil | — | — |
| content | | | | | | | | |
| b) Single | NMT 0.100% | Nil | — | — | Nil | Nil | — | — |
| largest | | | | | | | | |
| individual | | | | | | | | |
| Impurities | | | | | | | | |
| c) Total | NMT 0.500% | Nil | — | — | Nil | Nil | — | — |
| other | | | | | | | | |
| Impurities | | | | | | | | |
| d) Total | NMT 0.700% | Nil | — | — | Nil | Nil | — | — |
| Impurities | | | | | | | | |
| Assay | 95-105% | 99.8% | 100.0% | 99.9% | 101.6% | 100.1% | 99.8% | 99.8% |
| Specification | | 40° C./75% RH | | 50° C. | | | | UV |
| Test | Requirement | 14 days | 28 days | 3 days | 7 days | 14 days | 28 days | 3 days |
| Description | White powder | White | White | White | White | White | White | White |
| | | powder | powder | powder | powder | powder | powder | powder |
| Impurities | | | | | | | | |
| a) Lactam | NMT 0.200% | Nil | Nil | — | — | Nil | Nil | — |
| content | | | | | | | | |
| b) Single | NMT 0.100% | Nil | Nil | — | — | Nil | Nil | — |
| largest | | | | | | | | |
| individual | | | | | | | | |
| Impurities | | | | | | | | |
| c) Total | NMT 0.500% | Nil | Nil | — | — | Nil | Nil | — |
| other | | | | | | | | |
| Impurities | | | | | | | | |
| d) Total | NMT 0.700% | Nil | Nil | — | — | Nil | Nil | — |
| Impurities | | | | | | | | |
| Assay | 95-105% | 99.5% | 99.6% | 99.6% | 99.5% | 97.8% | 98.1% | 100.0% |

— not tested

TABLE 29
Pre-formulation studies, Batch Number: GD5

| Specification | | | 25° C./60% RH | | | | 40° C./75% RH | |
|---|---|---|---|---|---|---|---|---|
| Test | Requirement | Initial | 3 days | 7 days | 14 days | 28 days | 3 days | 7 days |
| Description | White powder | White | White | White | White | White | White | White |
| | | powder | powder | powder | powder | powder | powder | powder |
| Impurities | | | | | | | | |
| a) Lactam | NMT 0.200% | Nil | — | — | Nil | Nil | — | — |
| content | | | | | | | | |
| b) Single | NMT 0.100% | Nil | — | — | Nil | Nil | — | — |
| largest | | | | | | | | |
| individual | | | | | | | | |
| Impurities | | | | | | | | |
| c) Total | NMT 0.500% | Nil | — | — | Nil | Nil | — | — |
| other | | | | | | | | |
| Impurities | | | | | | | | |
| d) Total | NMT 0.700% | Nil | — | — | Nil | Nil | — | — |
| Impurities | | | | | | | | |
| Assay | 95-105% | 99.1% | 99.3% | 99.2% | 100.6% | 100.3% | 99.1% | 98.9% |
| Specification | | 40° C./75% RH | | 50° C. | | | | UV |
| Test | Requirement | 14 days | 28 days | 3 days | 7 days | 14 days | 28 days | 3 days |
| Description | White powder | White | White | White | White | White | White | White |
| | | powder | powder | powder | powder | powder | powder | powder |
| Impurities | | | | | | | | |
| a) Lactam | NMT 0.200% | Nil | Nil | — | — | Nil | 0.034% | — |
| content | | | | | | | | |
| b) Single | NMT 0.100% | Nil | Nil | — | — | Nil | 0.008% | — |
| largest | | | | | | | | |
| individual | | | | | | | | |
| Impurities | | | | | | | | |
| c) Total | NMT 0.500% | Nil | Nil | — | — | Nil | Nil | — |
| other | | | | | | | | |
| Impurities | | | | | | | | |
| d) Total | NMT 0.700% | Nil | Nil | — | — | Nil | 0.042% | — |
| Impurities | | | | | | | | |
| Assay | 95-105% | 99.4% | 99.96% | 99.6% | 99.0% | 97.9% | 98.7% | 99.3% |

— not tested

TABLE 30
Pre-formulation studies, Batch Number: GD6

| Specification | | | 25° C./60% RH | | | | 40° C./75% RH | | | | 50° C. | | | |
|---|---|---|---|---|---|---|---|---|---|---|---|---|---|---|
| Test | Requirement | Initial | 3 days | 7 days | 14 days | 28 days | 3 days | 7 days | 14 days | 28 days | 3 days | 7 days | 14 days | 28 days |
| De- | White | White | White | White | White | White | White | White | White | White | White | White | White | White |
| scription | powder | powder | powder | powder | powder | powder | powder | powder | powder | powder | powder | powder | powder | powder |
| Impurities | | | | | | | | | | | | | | |
| a) Lactam | NMT | Nil | — | — | Nil | Nil | — | — | Nil | Nil | — | — | Nil | Nil |
| content | 0.200% | | | | | | | | | | | | | |
| b) Single | NMT | Nil | — | — | Nil | Nil | — | — | Nil | Nil | — | — | Nil | Nil |
| largest | 0.100% | | | | | | | | | | | | | |
| individual | | | | | | | | | | | | | | |
| Impurities | | | | | | | | | | | | | | |
| c) Total | NMT | Nil | — | — | Nil | Nil | — | — | Nil | Nil | — | — | Nil | Nil |
| other | 0.500% | | | | | | | | | | | | | |
| Impurities | | | | | | | | | | | | | | |
| d) Total | NMT | Nil | — | — | Nil | Nil | — | — | Nil | Nil | — | — | Nil | Nil |
| Impurities | 0.700% | | | | | | | | | | | | | |
| Assay | 95-105% | 98.4% | — | 98.2% | — | 98.0% | — | 98.1% | — | 97.9% | — | 98.2% | — | 98.1% |

— not tested

TABLE 31
Pre-formulation studies, Batch Number: GD7

| Specification | | | 25° C./60% RH | | | | 40° C./75% RH | | | | 50° C. | | | |
|---|---|---|---|---|---|---|---|---|---|---|---|---|---|---|
| Test | Requirement | Initial | 3 days | 7 days | 14 days | 28 days | 3 days | 7 days | 14 days | 28 days | 3 days | 7 days | 14 days | 28 days |
| De- | White | White | White | White | White | White | White | White | White | White | White | White | White | White |
| scription | powder | powder | powder | powder | powder | powder | powder | powder | powder | powder | powder | powder | powder | powder |
| Impurities | | | | | | | | | | | | | | |
| a) Lactam | NMT | Nil | — | — | Nil | Nil | — | — | Nil | Nil | — | — | Nil | Nil |
| content | 0.200% | | | | | | | | | | | | | |
| b) Single | NMT | Nil | — | — | Nil | Nil | — | — | 0.052% | 0.049% | — | — | Nil | Nil |
| largest | 0.100% | | | | | | | | | | | | | |
| individual | | | | | | | | | | | | | | |
| Impurities | | | | | | | | | | | | | | |
| c) Total | NMT | Nil | — | — | Nil | Nil | — | — | Nil | Nil | — | — | Nil | Nil |
| other | 0.500% | | | | | | | | | | | | | |
| Impurities | | | | | | | | | | | | | | |
| d) Total | NMT | Nil | — | — | Nil | Nil | — | — | 0.052% | 0.049% | — | — | Nil | Nil |
| Impurities | 0.700% | | | | | | | | | | | | | |
| Assay | 95-105% | 98.9% | — | 98.7% | — | 98.3% | — | 98.6% | — | 98.2% | — | 97.7% | — | 98.3% |

— not tested

TABLE 32
Pre-formulation studies, Batch Number: GL1

| Specification | | | 25° C./60% RH | | | | 40° C./75% RH | |
|---|---|---|---|---|---|---|---|---|
| Test | Requirement | Initial | 3 days | 7 days | 14 days | 28 days | 3 days | 7 days |
| Description | White powder | White | White | White | White | White | White | White |
| | | powder | powder | powder | powder | powder | powder | powder |
| Impurities | | | | | | | | |
| a) Lactam | NMT 0.200% | Nil | — | — | Nil | 0.010% | — | — |
| content | | | | | | | | |
| b) Single | NMT 0.100% | Nil | — | — | Nil | Nil | — | — |
| largest | | | | | | | | |
| individual | | | | | | | | |
| Impurities | | | | | | | | |
| c) Total | NMT 0.500% | Nil | — | — | Nil | Nil | — | — |
| other | | | | | | | | |
| Impurities | | | | | | | | |
| d) Total | NMT 0.700% | Nil | — | — | Nil | 0.010% | — | — |
| Impurities | | | | | | | | |
| Assay | 95-105% | 100.0% | 100.0% | 100.1% | 100.6% | 100.3% | 98.6% | 98.4% |
| Specification | | 40° C./75% RH | | 50° C. | | | | UV |
| Test | Requirement | 14 days | 28 days | 3 days | 7 days | 14 days | 28 days | 3 days |
| Description | White powder | White | White | White | White | White | White | White |
| | | powder | powder | powder | powder | powder | powder | powder |
| Impurities | | | | | | | | |
| a) Lactam | NMT 0.200% | Nil | 0.050% | — | — | Nil | Nil | — |
| content | | | | | | | | |
| b) Single | NMT 0.100% | Nil | Nil | — | — | Nil | 0.009% | — |
| largest | | | | | | | | |
| individual | | | | | | | | |
| Impurities | | | | | | | | |
| c) Total | NMT 0.500% | Nil | Nil | — | — | Nil | Nil | — |
| other | | | | | | | | |
| Impurities | | | | | | | | |
| d) Total | NMT 0.700% | Nil | 0.050% | — | — | Nil | 0.009% | — |
| Impurities | | | | | | | | |
| Assay | 95-105% | 98.9% | 99.3% | 99.9% | 98.9% | 98.8% | 98.7% | 100.6% |

— not tested

TABLE 33
Pre-formulation studies, Batch Number: GL2

| Specification | | | 25° C./60% RH | | | | 40° C./75% RH | |
|---|---|---|---|---|---|---|---|---|
| Test | Requirement | Initial | 3 days | 7 days | 14 days | 28 days | 3 days | 7 days |
| Description | White powder | White | White | White | White | White | White | White |
| | | powder | powder | powder | powder | powder | powder | powder |
| Impurities | | | | | | | | |
| a) Lactam | NMT 0.200% | Nil | — | — | Nil | Nil | — | — |
| content | | | | | | | | |
| b) Single | NMT 0.100% | Nil | — | — | Nil | Nil | — | — |
| largest | | | | | | | | |
| individual | | | | | | | | |
| Impurities | | | | | | | | |
| c) Total | NMT 0.500% | Nil | — | — | Nil | Nil | — | — |
| other | | | | | | | | |
| Impurities | | | | | | | | |
| d) Total | NMT 0.700% | Nil | — | — | Nil | Nil | — | — |
| Impurities | | | | | | | | |
| Assay | 95-105% | 97.5% | 97.5% | 100.6% | 97.6% | — | 95.8% | 101.2% |
| Specification | | 40° C./75% RH | | 50° C. | | | | UV |
| Test | Requirement | 14 days | 28 days | 3 days | 7 days | 14 days | 28 days | 3 days |
| Description | White powder | White | White | White | White | White | White | White |
| | | powder | powder | powder | powder | powder | powder | powder |
| Impurities | | | | | | | | |
| a) Lactam | NMT 0.200% | Nil | Nil | — | — | Nil | Nil | — |
| content | | | | | | | | |
| b) Single | NMT 0.100% | Nil | Nil | — | — | Nil | Nil | — |
| largest | | | | | | | | |
| individual | | | | | | | | |
| Impurities | | | | | | | | |
| c) Total | NMT 0.500% | Nil | Nil | — | — | Nil | Nil | — |
| other | | | | | | | | |
| Impurities | | | | | | | | |
| d) Total | NMT 0.700% | Nil | Nil | — | — | Nil | Nil | — |
| Impurities | | | | | | | | |
| Assay | 95-105% | 96.74% | — | 90.3% | 86.9% | 96.6% | — | 100.6% |

— not tested

TABLE 34
Pre-formulation studies, Batch Number: GL3

| Specification | | | 25° C./60% RH | | | | 40° C./75% RH | |
|---|---|---|---|---|---|---|---|---|
| Test | Requirement | Initial | 3 days | 7 days | 14 days | 28 days | 3 days | 7 days |
| Description | White powder | White | White | White | White | White | White | White |
| | | powder | powder | powder | powder | powder | powder | powder |
| Impurities | | | | | | | | |
| a) Lactam | NMT 0.200% | 0.095% | — | — | 0.100% | 0.140% | — | — |
| content | | | | | | | | |
| b) Single | NMT 0.100% | Nil | — | — | Nil | Nil | — | — |
| largest | | | | | | | | |
| individual | | | | | | | | |
| Impurities | | | | | | | | |
| c) Total | NMT 0.500% | Nil | — | — | Nil | Nil | — | — |
| other | | | | | | | | |
| Impurities | | | | | | | | |
| d) Total | NMT 0.700% | 0.095% | — | — | 0.100% | 0.140% | — | — |
| Impurities | | | | | | | | |
| Assay | 95-105% | 98.9% | 98.9% | 97.4% | 96.3% | 96.31% | 99.0% | 97.2% |
| Specification | | 40° C./75% RH | | 50° C. | | | | UV |
| Test | Requirement | 14 days | 28 days | 3 days | 7 days | 14 days | 28 days | 3 days |
| Description | White powder | White | White | White | White | White | White | White |
| | | powder | powder | powder | powder | powder | powder | powder |
| Impurities | | | | | | | | |
| a) Lactam | NMT 0.200% | 0.240% | 0.29% | — | — | 1.260% | 1.050% | — |
| content | | | | | | | | |
| b) Single | NMT 0.100% | Nil | Nil | — | — | Nil | Nil | — |
| largest | | | | | | | | |
| individual | | | | | | | | |
| Impurities | | | | | | | | |
| c) Total | NMT 0.500% | Nil | Nil | — | — | Nil | Nil | — |
| other | | | | | | | | |
| Impurities | | | | | | | | |
| d) Total | NMT 0.700% | 0.240% | 0.240% | — | — | 1.260% | 1.050% | — |
| Impurities | | | | | | | | |
| Assay | 95-105% | 96.2% | 96.1% | 99.0% | 97.3% | 95.8% | 95.9% | 99.2% |

— not tested

TABLE 35
Pre-formulation studies, Batch Number: GS1

| Specification | | | 25° C./60% RH | | | | 40° C./75% RH | |
|---|---|---|---|---|---|---|---|---|
| Test | Requirement | Initial | 3 days | 7 days | 14 days | 28 days | 3 days | 7 days |
| Description | White powder | White | White | White | White | White | White | White |
| | | powder | powder | powder | powder | powder | powder | powder |
| Impurities | | | | | | | | |
| a) Lactam | NMT 0.200% | Nil | — | — | Nil | Nil | — | — |
| content | | | | | | | | |
| b) Single | NMT 0.100% | Nil | — | — | Nil | Nil | — | — |
| largest | | | | | | | | |
| individual | | | | | | | | |
| Impurities | | | | | | | | |
| c) Total | NMT 0.500% | Nil | — | — | Nil | Nil | — | — |
| other | | | | | | | | |
| Impurities | | | | | | | | |
| d) Total | NMT 0.700% | Nil | — | — | Nil | Nil | — | — |
| Impurities | | | | | | | | |
| Assay | 95-105% | 105.2% | 104.3% | 101.9% | 104.2% | 101.2% | 97.5% | 101.5% |
| Specification | | 40° C./75% RH | | 50° C. | | | | UV |
| Test | Requirement | 14 days | 28 days | 3 days | 7 days | 14 days | 28 days | 3 days |
| Description | White powder | White | White | White | White | White | White | White |
| | | powder | powder | powder | powder | powder | powder | powde |
| Impurities | | | | | | | | |
| a) Lactam | NMT 0.200% | Nil | Nil | — | — | Nil | Nil | — |
| content | | | | | | | | |
| b) Single | NMT 0.100% | Nil | Nil | — | — | Nil | Nil | — |
| largest | | | | | | | | |
| individual | | | | | | | | |
| Impurities | | | | | | | | |
| c) Total | NMT 0.500% | Nil | Nil | — | — | Nil | Nil | — |
| other | | | | | | | | |
| Impurities | | | | | | | | |
| d) Total | NMT 0.700% | Nil | Nil | — | — | Nil | Nil | — |
| Impurities | | | | | | | | |
| Assay | 95-105% | 97.1% | 100.9% | 99.4% | 104.6% | 103.5% | 100.9% | 105.6% |

— not tested

TABLE 36
Pre-formulation studies, Blend I

| Specification | | | 25° C./60% RH | | | | 40° C./75% RH | | | | 50° C. | | | |
|---|---|---|---|---|---|---|---|---|---|---|---|---|---|---|
| Test | Requirement | Initial | 3 days | 7 days | 14 days | 28 days | 3 days | 7 days | 14 days | 28 days | 3 days | 7 days | 14 days | 28 days |
| De- | White | White | White | White | White | White | White | White | White | White | White | White | White | White |
| scription | powder | powder | powder | powder | powder | powder | powder | powder | powder | powder | powder | powder | powder | powder |
| Impurities | | | | | | | | | | | | | | |
| a) Lactam | NMT | Nil | — | — | Nil | Nil | — | — | Nil | 0.062% | — | — | 0.097% | 0.130% |
| content | 0.200% | | | | | | | | | | | | | |
| b) Single | NMT | Nil | — | — | Nil | Nil | — | — | Nil | 0.007% | — | — | Nil | Nil |
| largest | 0.100% | | | | | | | | | | | | | |
| individual | | | | | | | | | | | | | | |
| Impurities | | | | | | | | | | | | | | |
| c) Total | NMT | Nil | — | — | Nil | Nil | — | — | Nil | Nil | — | — | Nil | Nil |
| other | 0.500% | | | | | | | | | | | | | |
| Impurities | | | | | | | | | | | | | | |
| d) Total | NMT | Nil | — | — | Nil | Nil | — | — | Nil | 0.069% | — | — | 0.097% | 0.130% |
| Impurities | 0.700% | | | | | | | | | | | | | |
| Assay | 95-105% | 102.5% | — | 102.0% | 101.9% | 101.95 | — | 101.8% | 101.4% | 101.1% | — | 101.9% | 101.2% | 101.9% |

— not tested

TABLE 37
Pre-formulation studies, Blend II

| Specification | | | 25° C./60% RH | | | | 40° C./75% RH | | | | 50° C. | | | |
|---|---|---|---|---|---|---|---|---|---|---|---|---|---|---|
| Test | Requirement | Initial | 3 days | 7 days | 14 days | 28 days | 3 days | 7 days | 14 days | 28 days | 3 days | 7 days | 14 days | 28 days |
| De- | White | White | White | White | White | White | White | White | White | White | White | White | White | White |
| scription | powder | powder | powder | powder | powder | powder | powder | powder | powder | powder | powder | powder | powder | powder |
| Impurities | | | | | | | | | | | | | | |
| a) Lactam | NMT | Nil | — | — | Nil | Nil | — | — | Nil | Nil | — | — | Nil | 0.030% |
| content | 0.200% | | | | | | | | | | | | | |
| b) Single | NMT | Nil | — | — | Nil | 0.007% | — | — | Nil | Nil | — | — | Nil | Nil |
| largest | 0.100% | | | | | | | | | | | | | |
| individual | | | | | | | | | | | | | | |
| Impurities | | | | | | | | | | | | | | |
| c) Total | NMT | Nil | — | — | Nil | Nil | — | — | Nil | Nil | — | — | Nil | Nil |
| other | 0.500% | | | | | | | | | | | | | |
| Impurities | | | | | | | | | | | | | | |
| d) Total | NMT | Nil | — | — | Nil | 0.007% | — | — | Nil | Nil | — | — | Nil | 0.030% |
| Impurities | 0.700% | | | | | | | | | | | | | |
| Assay | 95-105% | 102.8% | — | 102.5% | 102.4% | 101.1% | — | 102.2% | 101.8% | 101.1% | — | 102.0% | 101.7% | 102.6% |

— not tested

TABLE 38
Pre-formulation studies, Neurontin ® Capsules 400 mg, Batch Number: 0015077

| Specification | | | 25° C./60% RH | | | | 40° C./75% RH | | | | 50° C. | | | |
|---|---|---|---|---|---|---|---|---|---|---|---|---|---|---|
| Test | Requirement | Initial | 3 days | 7 days | 14 days | 28 days | 3 days | 7 days | 14 days | 28 days | 3 days | 7 days | 14 days | 28 days |
| De- | White | White | White | White | White | White | White | White | White | White | White | White | White | White |
| scription | powder | powder | powder | powder | powder | powder | powder | powder | powder | powder | powder | powder | powder | powder |
| Impurities | | | | | | | | | | | | | | |
| a) Lactam | NMT | Nil | — | — | 0.040% | Nil | — | — | 0.046% | Nil | — | — | 0.058% | Nil |
| content | 0.200% | | | | | | | | | | | | | |
| b) Single | NMT | Nil | — | — | Nil | Nil | — | — | Nil | Nil | — | — | Nil | Nil |
| largest | 0.100% | | | | | | | | | | | | | |
| individual | | | | | | | | | | | | | | |
| Impurities | | | | | | | | | | | | | | |
| c) Total | NMT | Nil | — | — | Nil | Nil | — | — | Nil | Nil | — | — | Nil | Nil |
| other | 0.500% | | | | | | | | | | | | | |
| Impurities | | | | | | | | | | | | | | |
| d) Total | NMT | Nil | — | — | 0.040% | Nil | — | — | 0.046% | Nil | — | — | 0.058% | Nil |
| Impurities | 0.700% | | | | | | | | | | | | | |
| Assay | 95-105% | 102.7% | — | 102.4% | 102.5% | — | — | 102.1% | 101.8% | — | — | 101.9% | 101.9% | — |

— not tested

TABLE 39
Pre-formulation studies, Drug Substance, Gabapentin Lot number: R 90562

| Specification | | | 25° C./60% RH | | | | 40° C./75% RH | | | | 50° C. | | | |
|---|---|---|---|---|---|---|---|---|---|---|---|---|---|---|
| Test | Requirement | Initial | 3 days | 7 days | 14 days | 28 days | 3 days | 7 days | 14 days | 28 days | 3 days | 7 days | 14 days | 28 days |
| De- | White | White | White | White | White | White | White | White | White | White | White | White | White | White |
| scription | powder | powder | powder | powder | powder | powder | powder | powder | powder | powder | powder | powder | powder | powder |
| Impurities | | | | | | | | | | | | | | |
| a) Lactam | NMT | Nil | — | — | Nil | Nil | — | — | Nil | Nil | — | — | 0.004% | 0.004% |
| content | 0.200% | | | | | | | | | | | | | |
| b) Single | NMT | Nil | — | — | Nil | Nil | — | — | Nil | Nil | — | — | Nil | Nil |
| largest | 0.100% | | | | | | | | | | | | | |
| individual | | | | | | | | | | | | | | |
| Impurities | | | | | | | | | | | | | | |
| c) Total | NMT | Nil | — | — | Nil | Nil | — | — | Nil | Nil | — | — | Nil | Nil |
| other | 0.500% | | | | | | | | | | | | | |
| Impurities | | | | | | | | | | | | | | |
| d) Total | NMT | Nil | — | — | Nil | Nil | — | — | Nil | Nil | — | — | Nil | Nil |
| Impurities | 0.700% | | | | | | | | | | | | | |
| Asay | 95-105% | 99.4% | — | 98.6% | 98.6% | 98.51% | — | 98.53% | 98.4% | 97.79% | — | 98.5% | 98.5% | 98.1% |

— not tested

### Preformulation Conclusions

The excipient compatibility study reveals that commonly used pharmaceutical excipients are compatible with gabapentin. The excipients studied do not adversely affect the stability of gabapentin when stored at 25° C./60% RH and 40° C./75% RH.

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention. It is intended, therefore, that the invention be defined by the scope of the claims that follow and that such claims be interpreted as broadly as is reasonable.

What we claim is:

1. A pharmaceutical composition of gabapentin wherein lactam level remains below 0.5 % w/w after at least 2 years of storage at 25° C. and 60% relative humidity.

2. A pharmaceutical composition of gabapentin wherein lactam level remains below 0.5% w/w after at least 2 years storage at 30° C. and 60% relative humidity.

3. A pharmaceutical composition of gabapentin wherein lactam level remains below 0.5% w/w after at least 6 months storage at 40° C. and 75% relative humidity.

4. A pharmaceutical composition of gabapentin comprising less than 0.5% w/w of lactam after storage conditions selected from the ranges consisting of: storage for at least 3 years at 25° C. and 60% relative humidity, storage for at least 2 years at 25 to 30° C. and 60% relative humidity, and storage for at least 6 months at 40° C. and 75% relative humidity.

5. The pharmaceutical composition according to claim 4, further comprising at least one excipient selected from the group consisting of dibasic calcium phosphate, tribasic calcium phosphate, calcium sulphate, mannitol, microcrystalline cellulose, starch IP, lactose, magnesium stearate, steric acid, colloidal silicon dioxide and sodium lauryl sulphate.

6. The pharmaceutical composition according to claim 5, comprising microcrystalline cellulose as the excipient.

7. The pharmaceutical composition according to claim 5, comprising magnesium stearate and microcrystalline cellulose as excipients.

8. The pharmaceutical composition according to claim 4, further comprising one or more of a diluent, a lubricant and a solubilizer.

9. The pharmaceutical composition according to claim 8, comprising at least one diluent selected from the group consisting of dibasic calcium phosphate, tribasic calcium phosphate, calcium sulphate, mannitol, microcrystalline cellulose, starch IP and lactose.

10. The pharmaceutical composition according to claim 8, comprising at least one lubricant selected from the group consisting of magnesium stearate, steric acid or colloidal silicon dioxide.

11. The pharmaceutical composition according to claim 8, comprising sodium lauryl sulphate as solubilizer.

12. The pharmaceutical composition according to claim 8, comprising microcrystalline cellulose as diluent.

13. The pharmaceutical composition according to claim 8, comprising magnesium stearate as lubricant.

14. The pharmaceutical composition according to claim 8, comprising microcrystalline cellulose as diluent and magnesium stearate as lubricant.

15. The pharmaceutical composition according to claim 8, comprising the composition in the form of a capsule for oral use.

16. The pharmaceutical composition according to claim 15, wherein the capsule comprises hard gelatin.

17. The capsule according to claim 16, further comprising at least one additive selected from the group consisting of methyl hydroxyl benzoate, propyl hydroxyl benzoate, titanium oxide, yellow iron oxide, and red iron oxide, in any suitable combination.

18. The pharmaceutical composition according to claim 4, wherein the stability of gabapentin is independent of the mineral acid anion content.

19. The pharmaceutical composition according claim 18, wherein the content of mineral acid anion is less than 70 ppm.

20. The pharmaceutical composition according to claim 18, wherein the content of mineral acid anion is less than 50 ppm.

21. The pharmaceutical composition according to claim 18, wherein the content of mineral acid anion is less than 30 ppm.

22. The pharmaceutical composition of claim 18, wherein the content of the mineral acid is in the range of 20 to 70 ppm.

23. The pharmaceutical composition of claim 18, wherein the content of the mineral acid is in the range of 20 to 50 ppm.

24. The pharmaceutical composition of claim 18, wherein the content of the mineral acid is in the range of 20 to 30 ppm.
